(19) United States
(12) Patent Application Publication
(10) Pub. No.: US 2013/0123271 A1
(43) Pub. Date: May 16, 2013

(54) HETEROCYCLIC COMPOUNDS, THEIR PREPARATION AND THERAPEUTIC APPLICATION

(71) Applicants: David Middlemiss, Paris (FR); Caroline Leriche, Paris (FR)

(72) Inventors: David Middlemiss, Paris (FR); Caroline Leriche, Paris (FR)

(73) Assignee: FOVEA PHARMACEUTICALS, Paris (FR)

(21) Appl. No.: 13/805,479

(22) PCT Filed: Jun. 22, 2011
(86) PCT No.: PCT/EP2011/060445

§ 371 Date: Dec. 19, 2012

(30) Foreign Application Priority Data
Jun. 22, 2010 (EP) 10305665.1

(51) Int. Cl. C07D 471/04; C07D 401/12; C07D 239/84

(52) U.S. Cl. CPC C07D 471/04; C07D 239/84; C07D 401/12
USPC 514/253.04; 546/119; 514/303; 544/322; 514/256; 544/362; 544/292; 514/266.4

(57) ABSTRACT

The invention is directed to certain novel compounds, methods for producing them and methods for treating or ameliorating a kinase-mediated disorder.

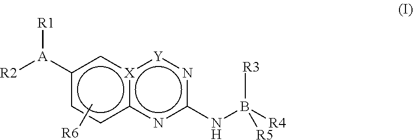

The invention is directed to certain novel compounds, methods for producing them and methods for treating or ameliorating a disorder involving tyrosine kinase dysregulation such as disorder associated with increased vascular permeability or angiogenesis. More particularly, this invention is directed to substituted triazolopyridine compounds useful as selective kinase inhibitors, methods for producing such compounds and methods for treating, preventing or ameliorating a kinase-mediated disorder. In particular, the methods relate to treating or ameliorating a disorder involving tyrosine kinase dysregulation including cardiovascular diseases, diabetes, diabetes-associated disorders, inflammatory diseases, immunological disorders, cancer and diseases of the eye such as retinopathies, macular degeneration or other vitreoretinal diseases, and the like.

Passage of fluid and cells out of blood vessels is a significant contributing factor to inflammation, tissue injury, oedema and death in a variety of circumstances. These include ischemic injury, toxic shock, burns, trauma, allergic and immune reactions. Vascular permeability is regulated in part by cell-cell adhesions between endothelial cells. The endothelial cell monolayer lining the vasculature forms a barrier that maintains the integrity of the blood fluid compartment, but permits passage of soluble factors and leukocytes in a regulated manner. Dysregulation of this process results in vascular leakage into surrounding tissues, which accompanies the inflammation associated with pathological oedematous conditions. Vascular permeability is a finely-tuned function that can positively contribute to protective immune responses and wound healing; however, in a number of pathological situations, massive and/or chronic leakage of fluid as well as migration of immune cells into tissues can have serious, and sometimes, life-threatening consequences.

Abnormal retinal vascular permeability leading to oedema in the area of the macula is the leading cause of vision loss in diseases such as diabetic retinopathy, exudative macular degeneration, retinal vascular occlusions, and inflammatory and neoplastic conditions. Although a variety of disease processes may lead to increased vascular permeability through different mechanisms, the cytokine VEGF is known to play a major role as inducer of vascular leakage. VEGF was first described as a potent vascular permeability factor (VPF) secreted by tumour cells that stimulated a rapid and reversible increase in microvascular permeability. Increased vascular permeability in ischemic retinopathies and possibly also in exudative macular degeneration and uveitis, for example, correlated with VEGF levels and VEGF antagonists have been successfully used to reduce retinal/macular oedema in neovascular eye diseases such as age-related macular degeneration leading to stabilization or even improvement of visual acuity in a subset of affected patients. The way by which VEGF induces vascular permeability has recently been unravelled and it has been shown that VEGF-induced vascular leakage is mediated by cytoplasmic protein kinase members of the Src proto oncogene family.

Protein kinases play a central role in the regulation and maintenance of a wide variety of cellular processes and cellular functions. For example, kinase activity acts as a molecular switch regulating cell proliferation, activation, and/or differentiation. It is now widely accepted that many diseases result from abnormal cellular responses triggered by overactive protein kinase-mediated pathways.

Src kinases form a family of membrane-attached non receptor-dependent tyrosine kinases encompassing eight members in mammals: Src, Fyn, Yes, Fgr, Lyn, Hck, Lck, and Blk which have important roles in receptor signalling and cellular communication. While most Src kinases are broadly expressed (i.e. Src, Fyn, Yes), certain members of the family such as Hck, Blk or Lck exhibit a restricted expression. Src kinases play a pivotal role as membrane-attached molecular switches that link a variety of extracellular cues to intracellular signalling pathways. This is the basis for the involvement of Src kinases in cell proliferation and differentiation as well as cell adhesion and migration.

It has been well-documented that Src protein levels and Src kinase activity are significantly elevated in human cancers including breast cancers, colon cancers, pancreatic cancers, certain B-cell leukemias and lymphomas, gastrointestinal cancer, non-small cell lung cancers, bladder cancer, prostate and ovarian cancers, melanoma and sarcoma. Thus, it has been anticipated that blocking signalling through the inhibition of the kinase activity of Src will be an effective means of modulating aberrant pathways that drive oncologic transformation of cells.

Similarly, it is well documented that Src-family kinases are also important for signalling downstream of immune cell receptors. Fyn, like Lck, is involved in TCR signalling in T cells. Hck and Fgr are involved in Fcy receptor signalling leading to neutrophil activation. Lyn and Src also participate in Fcy receptor signaling leading to release of histamine and other allergic mediators. These findings suggest that Src family kinase inhibitors may be useful in treating allergic diseases and asthma.

In accordance with the effect of VEGF on vascular permeability, several reports support a role of Src kinase in the development of oedema. For instance, Src but not Fyn deficiency or blockade of Src reduced brain oedema by about 55% following permanent cerebral ischemia in mice. Recently, PP1, a Src tyrosine kinase inhibitor was found to decrease oedema, to decrease breakdown of the brain-blood barrier (BBB), to reduce expression of VEGF. Similarly, Scheppke et al. have shown that Src kinases are critical mediators of VEGF- and ischemia-induced retinal vascular leakage.

Furthermore, Src tyrosine kinases fully mediate VEGF receptor signalling in vascular endothelial cells. Thus, activation of Src kinases resulting from stimulation of VEGF receptor or other growth factor located on endothelial cells or progenitors triggers angiogenesis, a response which can be deleterious in retinal and corneal diseases and which markedly contributes to tumor development and metastasis migration.

Several classes of compounds have been disclosed that modulate or, more specifically, inhibit kinase activity as potential treatments of kinase-mediated disorders, particularly cancer.

For example, WO2001038315 describes aminoquinazolines as inhibitors of cyclin-dependent kinases.

WO2008068507 describes pyridinylquinazolines as Raf serine/threonine kinase inhibitors for treating cancer.

WO2008079988 describes quinazolines as PDK1 kinase inhibitors for treating proliferative diseases such as cancer.

WO2006118256 describes quinazoline derivatives as p38MAPK inhibitors for inhalation and for treating various inflammatory diseases and cancer.

WO2006039718 describes aryl nitrogen-containing bicyclic compounds for use in treating protein kinase-mediated disease, including inflammation, cancer and related conditions.

WO2005037285 describes 2,6-disubstituted bicyclic heterocycles as Raf serine/threonine kinase inhibitors for treating disorders such as cancer.

WO2009046448 describes P13 kinase activity modulators having substituted aminoquinazoline on the pyrimidine part of the quinazoline bicycle.

WO2009084695 describes aminoquinazoline derivatives substituted by two non-aromatic substituents.

WO2008020203 describes aminoquinazoline derivatives substituted by pyridine on the phenyl part of the quinazoline bicycle and having B-Raf inhibiting activity.

U.S.20100093698 describes aminotriazolopyridines derivatives substituted in position 5 and having Syk kinase inhibition activity.

WO2004065378 describes 2-aminopyridines as cdk4 inhibitors for treating cell proliferative disorders such as cancer, atherosclerosis and restenosis.

Interestingly, WO2006024034 describes heterocyclic compounds derived from benzotriazine, triazines, triazoles and oxadiazoles, such as benzotriazine compounds (WO2005096784) or pyrimidine compounds (WO2006101977) which are capable of inhibiting kinases, such as members of the Src kinase family. Nevertheless, these drugs while they are claimed as potentially useful as for treatment of various ophthalmological diseases (e.g. age-related macular degeneration, diabetic retinopathy, diabetic macular oedema, cancer, and glaucoma) are lipophilic and water insoluble (see WO2006133411). According to the inventors of WO2006133411, these specific properties are particularly advantageous, particularly for ophthalmic uses, since these drugs being insoluble in water (water solubility of less than about 0.1 mg/mL at a pH range of 4-8) possess high efficiency of loading and negligible leakage due to high partitioning of the drug into the liposome used for delivering them compared to the water.

WO 2010076238 describes mono-substituted aminoquinazoline derivatives having a good 1050 against src and lyn kinases.

Src kinases inhibitors described in U.S.2005/0245524 are bright red in colour and very insoluble in formulations suitable for delivery by eye drops. These two parameters represent an important drawback for the compounds disclosed in U.S.2005/0245524.

The eye is a tightly protected organ. In this respect, treating diseases of the back-of-the-eye is probably the most difficult and challenging task of drug discovery as evidenced by the paucity of therapeutic options. One of the most convenient and safest form of drug delivery to the eye is eye drops, since it is non invasive, does not require medical assistance and requires small volumes of drug solution. However, in order to be suitable for topical instillation, molecules have to be potent enough towards their molecular target, to present physico-chemical properties allowing crossing of cell membranes, and to be sufficiently soluble in aqueous medium to be applied as solution onto the cornea. In addition, it is crucial that such drug molecules are as colourless as possible to prevent staining of ocular tissue which ultimately may interfer with vision. Additionally, due to the multiple cross reactivity between kinases, it is highly desirable that said drug molecules inhibit the targeted kinases with a high degree of selectivity.

A feature of the present invention is to provide novel compounds which have increased water solubility compared to competitors.

Another feature of the present invention is to provide compounds that are highly potent, particularly towards src kinase inhibitors.

Another feature of the present invention is to provide compounds which are useful for treating, preventing or ameliorating a disorder, including an ophthalmic disorder, involving tyrosine kinase dysregulation such as for example disorder associated with increased vascular permeability or angiogenesis.

Another feature of the present invention is to provide compounds which are colourless or almost colourless, especially in solution.

Additional features and advantages of the present invention will be set forth in part in the description that follows, and in part will be apparent from the description, or may be learned by practice of the present invention. The objectives and other advantages of the present invention will be realized and attained by means of the elements and combinations particularly pointed out in the description and appended claims.

The invention relates to compound of the general formula below:

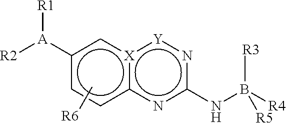

wherein
A is an aryl, an heterocycloalkyl, a —N-aryl, a —O-aryl, an heteroaryl, or a partially saturated heterocycloalkyl;
B is an heteroaryl or an aryl;
R1 and R2 are linked on a cycle and represent independently from each other:

—H,

—OH,

an halogen atom,

—O(C₁C₆)alkyl,

(C₁-C₆)alkyle,

—(CH₂)ₙOH,

—NH₂,

N-oxide wherein the nitrogen atom belongs to A,

with the provisio that R1 and R2 can both be hydrogen atoms only when A is a heterocycloalkyl, a —O-aryl, an heteroaryl or a partially substituted heterocycloalkyle;
R3, R4 and R5 are, independently from each other,

—H,

—(CH₂)ₙOH,

—O(C₁C₆)alkyl,

—(CH₂)ₙ—CO-heterocycloalkyl,

—OH,

-heterocycloalkyl-(CH₂)ₙ—OH,

-(C₁-C₆)alkyl,

—(CH₂)ₙ-heterocycloalkyl,

—(CH₂)ₙ-heterocycloalkyl-(CH₂)ₙ—OH,

—O—(CH₂)ₙ-heterocycloalkyl,

N-oxide wherein the nitrogen atom belongs to B,

—O—(CH₂)ₙ—CO-heterocycloalkyl,

—O—(CH₂)ₙ—OH,

—O(C₁C₆)alkyl-NR7R8,

—(C₁C₆)alkyl-NR7R8,

with the provisio that when A and B are aryl, at least two of R3, R4 and R5 are not hydrogen;

R6 is H, —O(C₁C₆)alkyl, or (C₁C₆)alkyl;
R7 and R8 are independently from each other H or (C₁C₆)alkyl;
n is 1, 2 or 3;

### X is N or C; and

Y is C or a bond,
as well as a prodrug thereof.

According to one embodiment, the invention concerns compounds of formula (I) as well as a prodrug of compounds of formula (I):

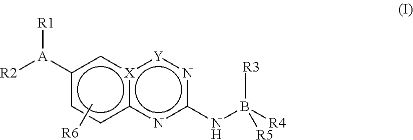

wherein
A is phenyl;
B is phenyl, pyridine, or pyrimidine
R1 and R2 represent independently from each other:

—H,

—OH,

a halogen atom,

with the provisio that R1 and R2 are not simultaneously hydrogen atoms;
R3, R4 and R5 are, independently from each other,

—H,

—(CH₂)ₙOH,

—O(C₁C₆)alkyl,

—CH₂)ₙ—CO-heterocycloalkyl,

—OH,

-heterocycloalkyl-(CH₂)ₙ—OH,

—(C₁-C₆) alkyl,

—(CH₂)ₙ-heterocycloalkyl,

—(CH₂)ₙ-heterocycloalkyl-(CH₂)ₙ—OH,

—O—(CH₂)ₙ-heterocycloalkyl,

N-oxide wherein the nitrogen atom belongs to B,

—O—(CH₂)ₙ—CO-heterocycloalkyl,

—O—(CH₂)ₙ—OH,

—O(C₁C₆)alkyl-NR7R8,

—(C₁C₆)alkyl-NR7R8,

or R3 and R4 form together with B a fused bicycle (such as for example indole or benzimidazole, optionally substituted by R5,

with the provisio that when A and B are aryl, at least two of R3, R4 and R5 are not hydrogen;

R6 is H, —O(C₁C₆)alkyl, or (C₁C₆)alkyl;
R7 and R8 are independently from each other H or an optionally substituted (C₁C₆)alkyl optionally forming a cycloalkyl;
n is 1, 2 or 3;

### X is N or C; and

Y is CH or a covalent bond.

This group of compounds of the Invention can be represented by formula (Ia) below:

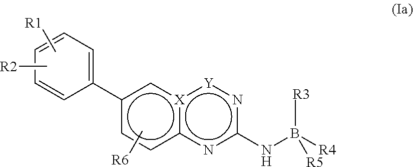

wherein R1, R2, R3, R4, R5, R6, R7, and R8 are as defined in the invention according to any embodiment or combination thereof.

In the context of the present specification, the terms defined below should be uderstood as having the meaning defined next to each term:

“a” and “an” are used in the sense that they mean “at least one”, “at least a first”, “one or more” or “a plurality” of the referenced compounds or steps, unless the context dictates otherwise. More specifically, “at least one” and “one or more” means a number which is one or greater than one, with a special preference for one, two or three;

“and/or” wherever used herein includes the meaning of “and”, “or” and “all or any other combination of the elements connected by said term”;

“about” or “approximately” means within 20%, preferably within 10%, and more preferably within 5% of a given value or range;

“comprising”, “containing” when used to define products, compositions and methods, is intended to mean that the products, compositions and methods include the referenced compounds or steps, but not excluding others;

“treatment” or “treating” encompasses prophylaxis and/or therapy. Accordingly the compositions and methods of the present invention are not limited to therapeutic applications and can be used in prophylaxis ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms;

“patient” and “subject in need thereof” are intended to mean any animal; such as a vertebrate, a member of the mammalian species and includes, but is not limited to, domestic animals (e.g. cows, hogs, sheep, horses, dogs, and cats), primates including humans. The terms “patient” “subject in need thereof” are in no way limited to a special disease status, it encompasses both patients who have already developed a disease of interest and patients who are not sick.

“therapeutically active compound” means any compound, optionally in a composition, that will elicit a desired biological response of a tissue, animal, or human, cell, or organ, for example.

“therapeutically effective amount” means any amount of a therapeutically active compound or composition.—“prodrug” means any compound administered in an inactive or significantly less active form than after its bioactivation. Once administered, the prodrug is metabolised in vivo into a therapeutically active compound (drug). This process is termed bioactivation. This bioactivation takes place in one or more steps, i.e. by providing one or more metabolites. A prodrug is usually not a therapeutically active compound itself and will usually not elicit in vitro the biological response of the corresponding therapeutically active compound after bioactivation. According to the present invention bioactivation takes place particularly in the cornea. This can be tested with Ussing chambers for example.

“halogen” means any one of fluoro, chloro, bromo or iodo;

“cycle”: means a cycloalkyl, a heterocycloalkyl, a heterocycloalkyl partially substituted, an aryl or a heteroaryl;

“cycloalkyl” means a saturated monocyclic carbocycle containing from 3 to 7 carbon atoms. Examples of monocyclic cycloalkyl radicals include cyclopropyl, cyclobutyl, cyclopentyl and the like;

“heterocycloalkyl” means a saturated mono- or bicyclic heterocycle having from 3 to 14 atoms, for example from 5 to 10 or from 5 to 6 atoms, and comprising at least one heteroatom selected from nitrogen, oxygen and sulphur. If the heterocycloalkyl contains more than one heteroatom, the heteroatoms can be identical or different. When substituted, the moiety can be substituted either on a carbon atom or on a heteroatom; similarly, the heterocycloalkyl can be attached to the rest of the molecule via a carbon atom or a heteroatom. Examples of heterocycloalkyl are pyrrolidine, piperidine, piperazine, morpholine and the like;

“heterocycloalkyl partially saturated” means an heterocycloalkyl comprising at least one double bond, but not enough double bonds to be considered as aromatic;

“aryl” includes mono- and bicyclic aromatic carbocycles. Examples of aryl include phenyl, 1-naphthyl, 2-naphthyl;

“heteroaryl” means an aromatic mono- or bicyclic aryl wherein each cycle comprises from 5 to 10 atoms, for example from 5 to 6 atoms, and comprising at least one heteroatom selected from nitrogen, oxygen and sulphur. If the heteroaryl contains more than one heteroatom, the heteroatoms can be identical or different. When substituted, the moiety can be substituted either on a carbon atom or on a heteroatom; similarly, the heteroaryl can be attached to the rest of the molecule via a carbon atom or a heteroatom. Examples of heteroaryl are pyridine, indole, benzofuran, oxazole, triazole, pyrimidine, pyrazole, indazole, benzimidazole and the like;

in “(C₁-C₆)”, the numbers define the possible number of atoms present in the chain or the cycle;

“alkyl” is a saturated aliphatic group, either linear or branched. For example, a C₁-C₆alkyl represents a carbonated chain comprising from 1 to 6 carbon atoms, either linear or branched, such as for example a methyl, ethyl, propyl, isopropyl, butyl, isobutyl, secbutyl, tertbutyl, pentyl.

The term “compound” herein is in general referring to compounds of formula I, or pharmaceutically acceptable prodrug, thereof.

Among the compounds of formula (I) that are subject matter of the invention, a first group is compounds of formula (II) below:

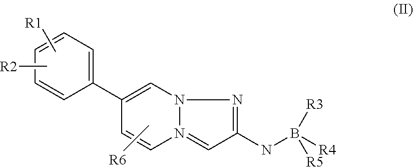

Among the compounds of formula (I) a second group is compounds of formula (III) below:

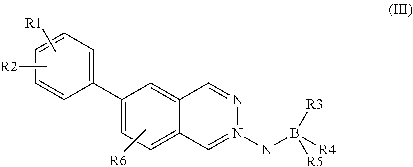

Among the compounds of formula (I) a third group is compounds of formula (IV) below:

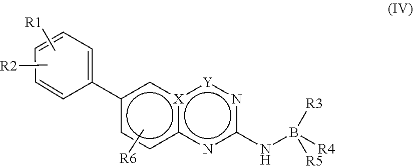

Among the compounds of the Invention, a fourth group of compounds is those having R1 and R2 in positions 3 and 6 of the phenyl ring.

Among the compounds of formula (I) a fifth group is compounds of formula (V) below:

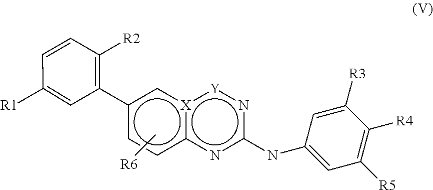

Among the compounds of formula (I) a sixth group is compounds of formula (VI) below:

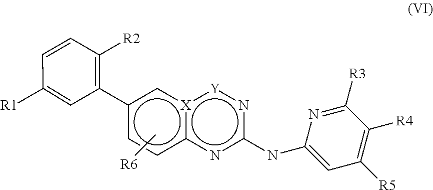

Among the compounds of formula (I) a seventh group is compounds of formula (VII) below:

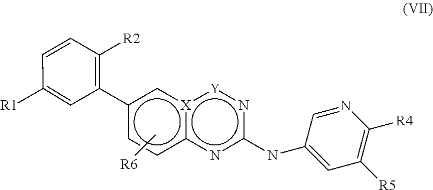

Among the compounds of formula (I) a eighth group is compounds of formula (VIII) below:

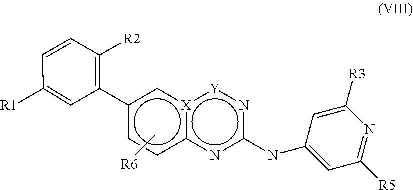

Among the compounds of formula (I) a ninth group is compounds of formula (IX) below:

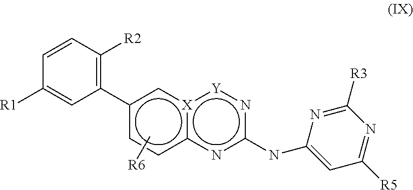

Among the compounds of formula (I) a tenth group is compounds of formula (X) below:

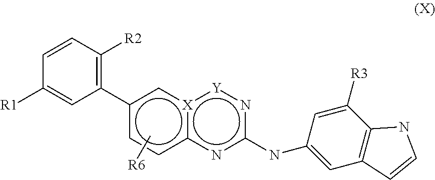

Among the compounds of formula (I) an eleventh group is compounds of formula (XI) below:

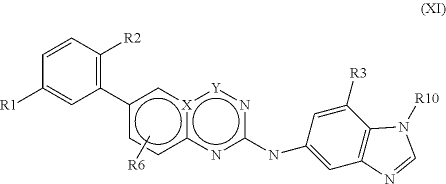

wherein R10 is

—H,

—(CH₂)ₙOH,

—O(C₁C₆)alkyl,

—(CH₂)ₙ-CO-heterocycloalkyl,

—OH,

-heterocycloalkyl-(CH₂)ₙ—OH,

—(C₁-C₆)alkyl,

—(CH₂)ₙ-heterocycloalkyl,

—(CH₂)ₙ-heterocycloalkyl-(CH₂)ₙ—OH,

—O—(CH₂)ₙ-heterocycloalkyl,

N-oxide wherein the nitrogen atom belongs to B,

—O—(CH₂)ₙ—CO-heterocycloalkyl,

—O—(CH₂)ₙ—OH,

—O(C₁C₆)alkyl-NR7R8, or

—(C₁C₆)alkyl-NR7R8.

In above formulae R1, R2, R3, R4, R5, R6, R7, and R8 are as defined in the invention according to any embodiment or combination thereof.

As apparent from skeletal formulae above, R6 is only bonded to the left ring of the bicycle.

In above formulae, a group of compounds is those wherein R1 is OH and R2 is a halogen atom. A particular halogen atom is chlorine or fluorine, and especially chlorine.

In above formulae, a group of compounds is those wherein R3, R4 and R5 represent independently from each other O-alkyl or hydroxyalkyl.

In above formulae, a group of compounds is those wherein R3, R4 and R5 represent independently from each other —CH₂OH, —O—CH₂—CH₂-heterocycloalkyl. For example, the heterocycloalkyl can be an optionally substituted pyrolidine, pyrrolidone, piperazine, or a morpholine. Particular substituents are —(C₁-C₆) alkyl, and —(C₁-C₆)hydroxyalkyl.

In above formulae, a group of compounds is those wherein X represents a carbon atom and Y represents CH.

In above formulae, a group of compounds is those wherein X represents a nitrogen, and Y represents a bond.

In above formulae, a group of compounds is those wherein R6 represents a hydrogen atom or CH₃. R6 is a hydrogen atom in a particular embodiment.

Compounds of the invention include those of the Examples herein, in particular the following, and their prodrugs:

compound 1: 4-Chloro-3-[2-(pyridin-4-ylamino)-[1,2,4]triazolo[1,5-a]pyridin-6-yl]-phenol

compound 2: 4-Chloro-3-[2-(pyridin-3-ylamino)-[1,2,4]triazolo[1,5-a]pyridin-6-yl]-phenol

compound 3: 4-Chloro-3-[2-(pyrimidin-5-ylamino)-[1,2,4]triazolo[1,5-a]pyridin-6-yl]-phenol

compound 4: 4-Chloro-3-[2-(5-hydroxymethyl-pyridin-3-ylamino)-[1,2,4]triazolo[1,5-a]pyridin-6-yl]-phenol

compound 5: 3-[2-(3,5-Bis-hydroxymethyl-phenylamino)-[1,2,4]triazolo[1,5-a]pyridin-6-yl]-4-chloro-phenol

compound 7: 4-Chloro-3-[2-(6-methoxy-pyridin-3-ylamino)-[1,2,4]triazolo[1,5-a]pyridin-6-yl]-phenol

compound 8: 4-Chloro-3-{2-[5-(2-pyrrolidin-1-yl-ethoxy)-pyridin-2-ylamino]-[1,2,4]triazolo[1,5-a]pyridin-6-yl}-phenol

compound 10: 4-Chloro-3-(2-{6-[4-(2-hydroxy-ethyl)-piperazin-1-yl]-pyridin-3-ylamino}-[1,2,4]triazolo[1,5-a]pyridin-6-yl)-phenol

compound 12: 4-Chloro-3-[2-(pyridin-2-ylamino)-[1,2,4]triazolo[1,5-a]pyridin-6-yl]-phenol

compound 13: 4-Chloro-3-[2-(2-hydroxymethyl-pyridin-4-ylamino)-[1,2,4]triazolo[1,5-a]pyridin-6-yl]-phenol

compound 14: 4-Chloro-3-[2-(6-hydroxymethyl-pyridin-3-ylamino)-[1,2,4]triazolo[1,5-a]pyridin-6-yl]-phenol

compound 16: 3-[2-(3,5-Bis-hydroxymethyl-phenylamino)-quinazolin-6-yl]-4-chloro-phenol

Compound 17: 4-Chloro-3-[2-(pyridin-3-ylamino)-quinazolin-6-yl]-phenol

Compound 18: 4-Chloro-3-[2-(1H-indol-6-ylamino)-[1,2,4]triazolo[1,5-a]pyridin-6-yl]-phenol

Compound 19: 4-[6-(2-Chloro-5-hydroxy-phenyl)-[1,2,4]triazolo[1,5-a]pyridin-2-ylamino]-pyridin-2-ol

Compound 20: 4-Chloro-3-[2-(2-methoxy-pyridin-4-ylamino)-[1,2,4]triazolo[1,5-a]pyridin-6-yl]-phenol

Compound 21: 4-Chloro-3-[2-(5-hydroxymethyl-pyridin-3-ylamino)-quinazolin-6-yl]-phenol

Compound 25: 4-Chloro-3-{2-[6-(2-pyrrolidin-1-yl-ethoxy)-pyridin-3-ylamino]-[1,2,4]triazolo[1,5-a]pyridin-6-yl}-phenol

Compound 26: 4-Chloro-3-{2-[5-(2-pyrrolidin-1-yl-ethoxy)-pyridin-3-ylamino]-[1,2,4]triazolo[1,5-a]pyridin-6-yl}-phenol

Compound 27: 4-Chloro-3-(2-{6-[4-(2-hydroxy-ethyl)-piperazin-1-yl]-2-methyl-pyrimidin-4-ylamino}-[1,2,4]triazolo[1,5-a]pyridin-6-yl)-phenol

Compound 28: 4-Chloro-3-(2-{3-[4-(2-hydroxy-ethyl)-piperazin-1-yl]-5-methyl-phenylamino}-[1,2,4]triazolo[1,5-a]pyridin-6-yl)-phenol

Compound 29: 4-Chloro-3-[2-(3,4,5-trimethoxy-phenylamino)-[1,2,4]triazolo[1,5-a]pyridin-6-yl]-phenol

Compound 30: 4-Chloro-3-{2-[3-(2-hydroxy-ethyl)-3H-benzoimidazol-5-ylamino]-[1,2,4]triazolo[1,5-a]pyridin-6-yl}-phenol

Compound 31: 4-Chloro-3-[2-(pyridin-3-ylamino)-[1,2,4]triazolo[1,5-a]pyridin-7-yl]-phenol

Compound 33: 4-Chloro-3-{2-[2-(2-pyrrolidin-1-yl-ethoxy)-pyridin-4-ylamino]-[1,2,4]triazolo[1,5-a]pyridin-6-yl}-phenol

Compound 34: 3-[2-(3,5-Bis-hydroxymethyl-phenylamino)-[1,2,4]triazolo[1,5-a]pyridin-7-yl]-4-chloro-phenol

Compound 35: 3-[2-(3,4-Bis-hydroxymethyl-phenylamino)-[1,2,4]triazolo[1,5-a]pyridin-6-yI]-4-chloro-phenol

Compound 36: 4-Chloro-3-[2-(3,4,5-trimethoxy-phenylamino)-quinazolin-6-y]-phenol

Compound 38: 4-Chloro-3-(2-{2-[4-(2-hydroxy-ethyl)-piperazin-1-yl]-pyridin-4-ylamino}-[1,2,4]triazolo[1,5-a]pyridin-6-yl)-phenol

Compound 39: 4-Chloro-3-{7-methoxy-2-[4-(2-pyrrolidin-1-yl-ethoxy)-phenylamino]-[1,2,4]triazolo[1,5-a]pyridin-6-yl}-phenol

Compound 40: 4-Chloro-3-[2-(6-methoxy-pyridin-3-ylamino)-quinazolin-6-yl]-phenol

Compound 41: 4-Chloro-3-(2-{4-[2-(1-oxy-pyrrolidin-1-yl)-ethoxy]-phenylamino}-[1,2,4]triazolo[1,5-a]pyridin-6-yl)-phenol

Compound 42: 4-Chloro-3-[2-(1 H-indol-6-ylamino)-quinazolin-6-yl]-phenol

Compound 43: 4-Chloro-3-[2-(2-hydroxymethyl-pyridin-4-ylamino)-quinazolin-6-yl]-phenol

Compound 44: 1-(2-{5-[6-(2-Chloro-5-hydroxy-phenyl)-[1,2,4]triazolo[1,5-a]pyridin-2-ylamino]-pyridin-2-yloxy}-ethyl)-pyrrolidin-2-one

Compound 45: 4-Chloro-3-{2-[1-(2-hydroxy-ethyl)-1H-benzoimidazol-5-ylamino]-quinazolin-6-yl}-phenol

Compound 46: 4-Chloro-3-(2-{3-[4-(2-hydroxy-ethyl)-piperazin-1-yl]-5-methyl-phenylamino}-quinazolin-6-yl)-phenol

Compound 47: 4-Chloro-3-(2-{3-[4-(2-hydroxy-ethyl)-piperazin-1-yl]-5-hydroxymethyl-phenylamino}-[1,2,4]triazolo[1,5-a]pyridin-6-yl)-phenol

Compound 48: Benzoic acid 4-chloro-3-(2-{3-[4-(2-hydroxy-ethyl)-piperazin-1-yl]-5-methyl-phenylamino}-[1,2,4]triazolo[1,5-a]pyridin-6-yl)-phenyl ester

Compound 49: Benzoic acid 4-chloro-3-(2-{3-[4-(2-hydroxy-ethyl)-piperazin-1-yl]-5-methyl-phenylamino}-quinazolin-6-yl)-phenyl ester

Compound 50: 4-Chloro-3-(2-{3-[4-(2-hydroxy-ethyl)-piperazin-1-yl]-5-hydroxymethyl-phenylamino}-quinazolin-6-yl)-phenol;

and any prodrug thereof.

A group of prodrugs is esters of compounds of above formulae, and in particular esters of benzoic acid with the phenol ring of above formulae (where R1 or/or R2 is —OH). Examples of prodrugs are:

Benzoic acid 4-chloro-3-(2-{3-[4-(2-hydroxy-ethyl)-piperazin-1-yl]-5-methyl-phenylamino}-[1,2,4]triazolo[1,5-a]pyridin-6-yl)-phenyl ester; and

Benzoic acid 4-chloro-3-(2-{3-[4-(2-hydroxy-ethyl)-piperazin-1-yl]-5-methyl-phenylamino}-quinazolin-6-yl)-phenyl ester.

According to another embodiment, the compounds of the Invention are either white or with a pale colour when in powder, and are uncoloured and transparent when in aqueous solution at active concentrations.

The compounds of the present invention act primarily on src kinase.

According to another embodiment, the compounds of the Invention are src kinase inhibitors.

According to another embodiment, particular compounds of the Invention have an IC50 towards Src of less than about 15 nM, advantageously less than about 10 nM, for example less than about 1 nM, less than about 0.9 nM, or even less than about 0.5 nM.

According to another embodiment, there are provided compositions including one or more compounds of the Invention and a pharmaceutically acceptable carrier or aqueous medium.

As used herein, the term “pharmaceutically acceptable” refers to carriers that do not produce an adverse, allergic or other unwanted reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such carriers for pharmaceutical active substances is well known in the art. Examples of suitable pharmaceutical carriers are described in “Remington's Pharmaceutical Sciences” by E. W. Martin. In a particular embodiment, the compounds of the Invention are formulated in accordance with routine procedures as a pharmaceutical composition adapted for administration to the eye. Supplementary active ingredients, such as anti-inflammatory agent, chemotherapeutic agent, anti-cancer agent, immunomodulatory agent, gene-based therapeutic vaccine, immunotherapy product, therapeutic antibody and/or protein kinase inhibitors can also be incorporated into the compositions.

According to one embodiment, the compounds of the present invention will be formulated for parenteral administration, e.g., formulated for injection via the intravenous, intramuscular, subcutaneous, or even intraperitoneal routes. The preparation of an aqueous composition that contains a compound or compounds of the Invention will be within the skill of those in the art, in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

According to a particular embodiment, the compounds of the present invention will be formulated for topical administration of the compounds of the Invention, especially for the treatment of ophthalmic disorders. The preparation of a composition that contains a compound or compounds of the Invention will be within the skill of those in the art, in light of the present disclosure. Typically, such compositions for topical administration can be prepared as ointment, gel or eye drops. The topical ophthalmic composition may further be an in situ gel formulation. Such a formulation comprises a gelling agent in a concentration effective to promote gelling upon contact with the eye or with lacrimal fluid in the exterior of the eye. Suitable gelling agents include, but are not limited to, thermosetting polymers such as tetra-substituted ethylene diamine block copolymers of ethylene oxide and propylene oxide (e.g., poloxamine); polycarbophil; and polysaccharides such as gellan, carrageenan (e.g., kappa-carrageenan and iota-carrageenan), chitosan and alginate gums. The phrase “in situ gellable” as used herein embraces not only liquids of low viscosity that form gels upon contact with the eye or with lacrimal fluid in the exterior of the eye, but also more viscous liquids such as semi-fluid and thixotropic gels that exhibit substantially increased viscosity or gel stiffness upon administration to the eye.

According to another embodiment, the compounds of the present invention will be formulated for oral administration of the compounds of the Invention. The preparation of a composition that contains a compound or compounds of the Invention will be within the skill of those in the art, in light of the present disclosure. Typically, such compositions for oral administration can be prepared as liquid solutions or suspensions, tablets, time release capsules and other solids for oral administration.

According to another embodiment, the compounds of the present invention will be formulated for intratumoral administration of the compounds of the Invention. The preparation of a composition that contains a compound or compounds of the Invention will be within the skill of those in the art, in light of the present disclosure. Typically, such compositions for intratumoral administration can be prepared as disclosed above for the other routes of administration.

According to another embodiment, the compounds of the present invention will be formulated for inhaled administration of the compounds of the Invention. The preparation of a composition that contains a compound or compounds of the Invention will be within the skill of those in the art, in light of the present disclosure. Typically, such compositions for inhalation can be prepared as disclosed above for the other routes of administration.

According to another particular embodiment, the compounds of the present invention will be combined with ophthalmologically acceptable preservatives, viscosity enhancers, penetration enhancers, buffers, sodium chloride, and water to form an aqueous, sterile ophthalmic suspension or solution. Ophthalmic solution formulations may be prepared by dissolving a compound in a physiologically acceptable isotonic aqueous buffer. Further, the ophthalmic solution may include an ophthalmologically acceptable surfactant to assist in dissolving the compound. Furthermore, the ophthalmic solution may contain an agent to increase viscosity, such as hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient can be combined with a preservative in an appropriate vehicle, such as, mineral oil, liquid lanolin, or white petrolatum. The compounds are preferably formulated as topical ophthalmic suspensions or solutions, with a pH of about 5 to 8, and more preferably from about 6.5 to about 7.5. The compounds will normally be contained in these formulations in an amount 0.001% to 5% by weight, but preferably in an amount of 0.025% to 2% by weight. Thus, for topical presentation 1 to 2 drops of these formulations would be delivered to the surface of the eye 1 to 4 times per day according to the discretion of a skilled clinician.

In another embodiment, there are provided methods of treating a disorder involving tyrosine kinase dysregulation such as disorder associated with increased vascular permeability or angiogenesis, including the administration of a therapeutically effective amount of one or more compound of the Invention to a subject in need of such treatment.

According to one embodiment, the said disorder involving tyrosine kinase dysregulation is a disorder associated with increased vascular permeability.

According to another embodiment, the said disorder involving tyrosine kinase dysregulation is a disorder associated with angiogenesis.

In particular embodiment, the disorder involving tyrosine kinase dysregulation is a disorder associated with a src kinase dysregulation.

According to one embodiment, the said disorder involving tyrosine kinase dysregulation is selected in the group consisting of myocardial infarction, stroke, congestive heart failure, an ischemia or reperfusion injury, trauma, cancer, oedema, arthritis or other arthropathy, transplant rejection, autoimmune disease, burn, or acute or adult respiratory distress syndrome (ARDS), or ophthalmic disorders such as retinopathy or vitreoretinal disease, diabetic retinopathy, macular oedema, including diabetic macular oedema, macular degeneration, glaucoma, vascular leakage syndrome, inflammatory disease, or oedema, for example.

In another embodiment, there are provided methods of treating an ophthalmic disorder associated with increased vascular permeability, including the administration of a therapeutically effective amount of one or more compound of the Invention to a subject in need of such treatment.

In another embodiment, there are provided methods of treating a subject having or at risk of having cancer including administering to the subject a therapeutically effective amount of one or more compound of the Invention thereby treating the subject.

In another embodiment, there are provided methods of treating a subject having or at risk of having oedema and/or angiogenesis including administering to the subject a therapeutically effective amount of one or more compound of the Invention, thereby treating the subject.

In another embodiment, there are provided methods of treating a subject having or at risk of having macular degeneration including administering to the subject a therapeutically effective amount of one or more compound of the Invention, thereby treating the subject.

In another embodiment, there are provided methods of treating a subject having or at risk of having diabetic retinopathy including administering to the subject a therapeutically effective amount of one or more compound of the Invention, thereby treating the subject.

In another embodiment, there are provided methods of treating a subject having or at risk of having macular oedema, including diabetic macular oedema, including administering to the subject a therapeutically effective amount of one or more compound of the Invention, thereby treating the subject.

In another embodiment, there are provided methods of treating a subject having or at risk of having glaucoma including administering to the subject a therapeutically effective amount of one or more compound of the Invention, thereby treating the subject.

In another embodiment, there are provided methods of treating a subject having or at risk of having retinopathy including administering to the subject a therapeutically effective amount of one or more compound of the Invention, thereby treating the subject.

In another embodiment, there are provided methods of treating a subject having or at risk of having vitreoretinal disease including administering to the subject a therapeutically effective amount of one or more compound of the Invention, thereby treating the subject.

In another embodiment, there are provided methods of treating a subject having or at risk of having inflammatory disease, including administering to the subject a therapeutically effective amount of one or more compound of the Invention, thereby treating the subject.

In yet another embodiment, there are provided methods of treating a disorder, including an ophthalmic disorder and cancer, associated with compromised vascular permeability including the administration of a therapeutically effective amount of one or more compound of the Invention in combination with an anti-inflammatory agent, chemotherapeutic agent, antitumoral agent, immunomodulatory agent, gene-based therapeutic vaccine, immunotherapy product, therapeutic antibody and/or a kinase inhibitor, to a subject in need of such treatment.

Administration of the compounds of the Invention, especially for ophthalmic applications, is preferably by topical administration. However, the invention is not limited to topical delivery in that it also includes for example intraocular and periocular injection, systemic delivery (e.g. oral or other parenteral route such as for example subcutaneous, intramuscular, intravenous administrations) or intratumoral delivery.

In yet another embodiment, there are provided methods of delivering a compound of the Invention to the back of the eye, the method including preparing a composition including a pharmaceutically effective amount of at least one compound of the Invention and delivering said composition to the eye of a subject in need of such delivery.

In yet another embodiment, there are provided methods of delivering a compound of the Invention intratumoraly, the method including preparing a composition including a pharmaceutically effective amount of at least one compound of the Invention and delivering said composition to the tumor of a subject in need of such delivery.

To prepare a composition of the Invention, and more specifically an ophthalmic composition or antitumoral composition, a therapeutically effective amount of one or more compound of the Invention is placed in a vehicle as is known in the art. For example, topical ophthalmic formulations containing steroids are disclosed in U.S. Pat. No. 5,041,434, whilst sustained release ophthalmic formulations of an ophthalmic drug and a high molecular weight polymer to form a highly viscous gel have been described in U.S. Pat. No. 4,271,143 and U.S. Pat. No. 4,407,792. Further GB 2007091 describes an ophthalmic composition in the form of a gel comprising an aqueous solution of a carboxyvinyl polymer, a water-soluble basic substance and an ophthalmic drug. Alternatively, U.S. Pat. No. 4,615,697, discloses a controlled release composition and method of use based on a bioadhesive and a treating agent, such as an anti-inflammatory agent.

The amount of the compounds of the Invention to be administered and its concentration in the compositions used in the method of the Invention depend upon the selected dissolving agent, delivery system or device, clinical condition of the patient, side effects and stability of the compound within the composition. Thus, the physician employs the appropriate preparation containing the appropriate concentration of the compounds of the Invention and selects the amount of formulation administered, depending upon clinical experience with a given patient or with similar types of patients.

In another embodiment, there are provided processes for making one or more compound of the Invention or a prodrug thereof.

There are multiple synthetic routes for the preparation of the compounds of the invention, but all rely on chemistry known to the synthetic organic chemist. Thus, compounds represented by Formula I can be synthesized according to procedures described in the literature and are well-known to one skilled in the art. Typical literature sources are “Advanced organic chemistry”, 4th Edition (Wiley), J March, “Comprehensive Organic Transformation”, 2nd Edition (Wiley), R. C. Larock, “Handbook of Heterocyclic Chemistry”, 2nd Edition (Pergamon), A. R. Katritzky), review articles such as found in “Synthesis”, “Acc. Chem. Res.”, “Chem. Rev”, or primary literature sources identified by standard literature searches online or from secondary sources such as “Chemical Abstracts” or “Beilstein”. Compounds of the invention can be synthesized by methods analogous to those exemplified in the Examples herein for certain representative compounds. Using the procedures described in the Examples section, and well known procedures, one skilled in the art can prepare the compounds disclosed herein.

In another embodiment, there are provided kit including packaging material and a composition contained within the packaging material, wherein the packaging material includes a label which indicates that the composition can be used for treatment of disorders associated with compromised vascular permeability and wherein the composition includes one or more compound of the Invention.

In another embodiment, there are provided kit including packaging material and a composition contained within the packaging material, wherein the packaging material includes a label which indicates that the composition can be used for treatment of disorders associated with compromised vascular permeability and selected from myocardial infarction, stroke, congestive heart failure, an ischemia or reperfusion injury, cancer, arthritis or other arthropathy, retinopathy or vitreoretinal disease, macular degeneration, autoimmune disease, vascular leakage syndrome, inflammatory disease, edema, transplant rejection, burn, or acute or adult respiratory distress syndrome (ARDS) and wherein the composition includes one or more compound of the Invention.

In one particular embodiment, there are provided kit including packaging material and a composition contained within the packaging material, wherein the packaging material includes a label which indicates that the composition can be used for treatment of ophthalmic disorders associated with compromised vascular permeability and wherein the composition includes one or more compounds of the Invention, or one or more prodrugs of a compound of the Invention.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

The present invention is not to be limited in scope by the specific embodiments described herein, which are intended for the purpose of exemplification only. Functionally equivalent products, formulations and methods are clearly within the scope of the invention as described herein.

The invention described herein may include one or more range of values (eg size, concentration etc). A range of values will be understood to include all values within the range, including the values defining the range, and values adjacent to the range which lead to the same or substantially the same outcome as the values immediately adjacent to that value which defines the boundary to the range.

The following examples are given to illustrate the preparation of compounds that are the subject of this invention but should not be construed as implying any limitations to the claims. The proton magnetic resonance spectrum of each compound of the Examples was consistent with the assigned structure.

### EXAMPLES 1—Synthesis of Compounds of General Formula (I)

1.1. General Method

Step A—Coupling of 7-Bromo-[1,2,4]triazolo[1,5-a]pyridin-2-ylamine or 6-Bromo-[1,2,4]triazolo[1,5-a]pyridin-2-ylamine to 1 eq of optionally substituted R1,R2-phenyl boronic acid in a polar solvent at —100 to 300° C., most preferably 50-150° C.

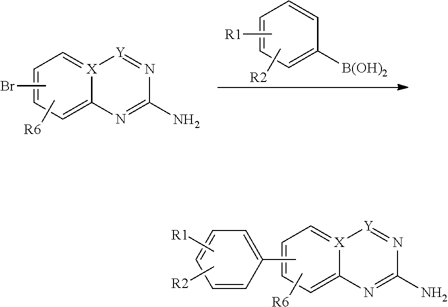

Step B—Coupling of (R3, R4, R5)-substituted bromo-phenyl to 1 eq of optionally substituted 7-phenyl (R1, R2 substituted)-[1,2,4]triazolo[1,5-a]pyridin-2-ylamine or 6-phenyl (R1, R2 substituted)-[1,2,4]triazolo[1,5-a]pyridin-2-ylamine in a polar solvent at −100° C. to 300° C., most preferably 50-150° C.

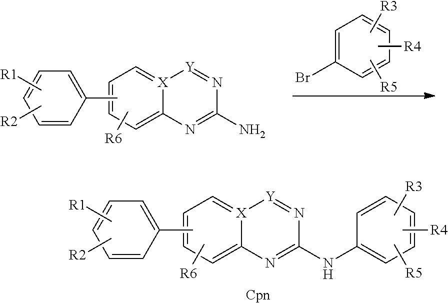

The compounds of the formula I and also the starting materials for their preparation, are prepared by methods as described in the examples or by methods known per se, as described in the literature (for example in standard works, such as Houben-Weyl, Methoden der Organischen Chemie [Methods of Organic Chemistry], Georg Thieme Verlag, Stuttgart; Organic Reactions, John Wiley & Sons, Inc., New York), to be precise under reaction conditions which are known and suitable for the said reactions. Use can also be made here of variants which are known per se, but are not mentioned here in greater detail.

The starting materials for the claimed process may, if desired, also be formed in situ by not isolating them from the reaction mixture, but instead immediately converting them further into the compounds of the formula I. On the other hand, it is possible to carry out the reaction stepwise.

Preferably, the reaction of the compounds is carried out in the presence of a suitable solvent, which is preferably inert under the respective reaction conditions. Examples of suitable solvents are hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, dimethylformamide (DMF) or N-methyl pyrrolidinone (NMP); nitriles, such as acetonitrile; sulfoxides, such as dimethyl sulfoxide (DMSO); nitro compounds, such as nitromethane or nitrobenzene; esters, such as ethyl acetate, or mixtures of the said solvents or mixtures with water. Polar solvents are in general preferred. Examples for suitable polar solvents are chlorinated hydrocarbons, alcohols, glycol ethers, nitriles, amides and sulfoxides or mixtures thereof. More preferred are amides, especially dimethylformamide (DMF).

As stated above, the reaction temperature is between about −100° C. and 300° C., depending on the reaction step and the conditions used.

Reaction times are generally in the range between some minutes and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 min and 48 hrs.

Every reaction step described herein can optionally be followed by one or more working up procedures and/or isolating procedures. Suitable such procedures are known in the art, for example from standard works, such as Houben-Weyl, Methoden der organischen Chemie [Methods of Organic Chemistry], Georg-Thieme-Verlag, Stuttgart). Examples for such procedures include, but are not limited to evaporating a solvent, distilling, crystallization, fractionised crystallization, extraction procedures, washing procedures, digesting procedures, filtration procedures, chromatography, chromatography by HPLC and drying procedures, especially drying procedures in vacuo and/or elevated temperature.

List of Abbreviations and Acronyms:

AcOH acetic acid, anh anhydrous, atm atmosphere(s), BOC tert-butoxycarbonyl CDI 1,1′-carbonyl diimidazole, conc concentrated, d day(s), dec decomposition, DMAC NN-dimethylacetamide, DMPU 1,3-dimethyl-3,4,5,6-tetrahydro-2(IH)-pyrimidinone, DMF NN-dimethylformamide, DMSO dimethylsulfoxide, DPPA diphenylphosphoryl azide, EDCI 1-(3-dimethylaminopropyl)-3-ethylcarbodiimide, EtOAc ethyl acetate, EtOH ethanol (100%), Et₂O diethyl ether, Et₃N triethylamine, h hour(s), MeOH methanol, pet. ether petroleum ether (boiling range 30-60° C.), temp. temperature, THF tetrahydrofuran, TFA trifluoroAcOH, Tf trifluoromethanesulfonyl.

The compounds of general formula I of the present invention can be prepared according to the procedures of the following Steps A and B above disclosed and the examples. In all preparative methods, all starting material is known or may easily be prepared from known starting materials.

1.2. Intermediates

In all preparative methods, all starting materials are known or may be prepared from known starting materials by the following general methods,

Either:

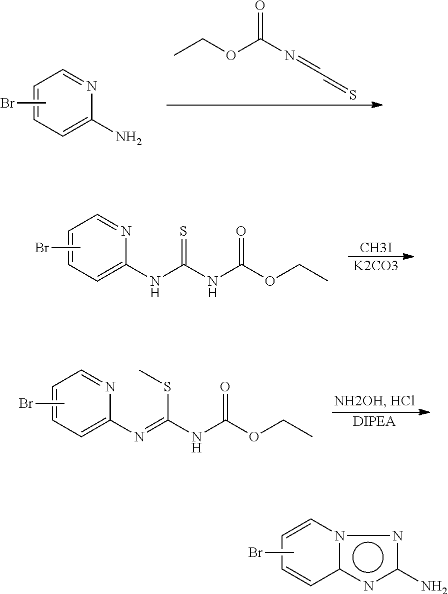

The compounds can be prepared by the general method, following procedures depicted in WO2007/095588 (Novartis).

Or:

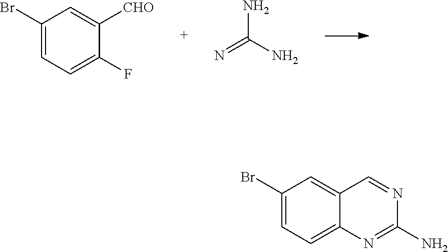

The compounds can be prepared by the general method, following procedures depicted in J. Heterocyclic Chem. 34, 385 (1997).

Method 1:

Synthesis of Intermediate 1: 6-(2-Chloro-5-methoxy-phenyl)-[1,2,4]triazolo[1,5-a]pyridin-2-ylamine

To a solution of 2-Chloro-5-methoxy-phenylboronic acid (3.38 g, 22.5 mmol, 1.5 eq), 6-Bromo-[1,2,4]triazolo[1,5-a]pyridin-2-ylamine (3.2 g, 15 mmol, 1 eq) and Na2CO3 (6.36 g, 60 mmol, 4 eq) in a mixture of 40 ml DMF/10 ml EtOH/10 ml H2O, was added 1.733 g (1.5 mmol, 0.1 eq) of tetrakis(triphenylphospine) palladium. The reaction was refluxed for 2 hours under argon. It was then cooled off to room temperature and the product was precipitated by water, filtered, rinsed with water, ether and pentane to give a pale yellow powder (3.21 g, 13 mmol, 90% yield).

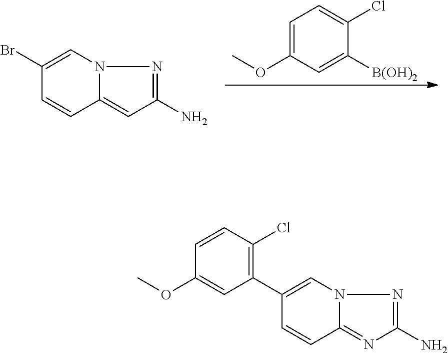 [SMILES: Nc1cc2ccc(Br)cn2n1 ; COc1ccc(Cl)c(B(O)O)c1 ; COc1ccc(Cl)c(-c2ccc3nc(N)nn3c2)c1]

Intermediate 2: 7-(2-Chloro-5-methoxy-phenyl)-[1,2,4]triazolo[1,5-a]pyridin-2-ylamine has been synthesized according to the method disclosed for Intermediate 1 starting from 7-Bromo-[1,2,4]triazolo[1,5-a]pyridin-2-ylamine.

Synthesis of intermediate 3: 3-(2-Amino-[1,2,4]triazolo[1,5-a]pyridin-6-yl)-4-chloro-phenol

To a suspension of 5.560 g (20.24 mmol, 1 eq) of 6-(2-Chloro-5-methoxy-phenyl)-[1,2,4]triazolo[1,5-a]pyridin-2-ylamine in 90 ml of dichloromethane cooled to 0° C. was added carefully 60 ml of a 1M solution of 1M BBr3. The solution is stirred for 2 hrs. The pH is then adjusted to pH8 by adding a sturated solution of NaHCO3. The precipitated product is filtered and washed with ether and dried to give 4.856 g (19 mmol, 92%) of a white powder.

Intermediate 4: 3-(2-Amino-[1,2,4]triazolo[1,5-a]pyridin-7-yl)-4-chloro-phenol has been synthesized according to the method disclosed for Intermediate 3 starting from 7-(2-Chloro-5-methoxy-phenyl)-[1,2,4]triazolo[1,5-a]pyridin-2-ylamine.

The compounds can also be prepared by the general method 2.

Method 2:

Synthesis of intermediate 5: 6-(2-Chloro-5-methoxy-phenyl)-quinazolin-2-ylamine

To a solution of 2-chloro-5-methoxy boronic acid (14.42 g, 77.34 mmol, 1.5eq), 6-Bromo-quinazolin-2-ylamine (11.55 g, 51.56 mmol, 1 eq) and Na2CO3 (21.86 g, 206.23 mmol, 4 eq) in a mixture of 120 ml DMF/30 ml EtOH/30 ml H2O, was added 2.311 g (5.16 mmol, 0.1 eq) of tetrakis(triphenylphospine) palladium. The reaction was refluxed (100° C.) for 2 hours under argon. It was then cooled off to room temperature to extract the product by DCM and brine. The product is then washed with water and ether, then dried to give 9.010 g (32 mmol, 61%) of a pale yellow powder.

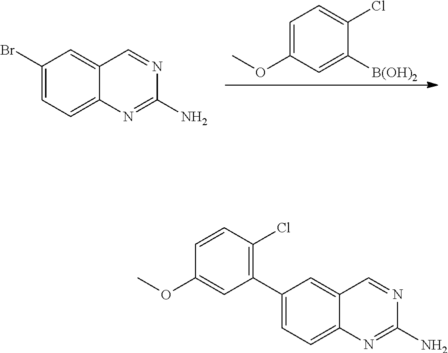 [SMILES: Nc1ncc2cc(Br)ccc2n1 ; COc1ccc(Cl)c(B(O)O)c1 ; COc1ccc(Cl)c(-c2ccc3nc(N)ncc3c2)c1]

Intermediate 6: (6-(2,6-Dimethyl-phenyl)-quinazolin-2-ylamine) has been synthesized according to the method disclosed for Intermediate 1.

Synthesis of intermediate 7: 3-(2-Amino-quinazolin-6-yl)-4-chloro-phenol

To a suspension of 9.010 g (31.53 mmol, 1 eq) of 6-(2-Chloro-5-methoxy-phenyl)-quinazolin-2-ylamine in 300 ml of dichloromethane cooled to 0° C. was added carefully 95 ml of a 1M solution of 1M BBr3. The solution is stirred for 16 hrs. The pH is then adjusted to pH8 by adding a sturated solution of NaHCO3. The precipitated product is filtered and washed with ether and dried to give 7.596 g (27.96 mmol, 89%) of a pale yellow powder.

1.3. Compounds of the Invention

Synthesis of compound of the Invention N° 5—Method 1

To 49 mg (0.05 mmol, 0.03 eq) of Pd₂(dba)₃, 16 mg (0.03 mmol, 0.02 eq) of 5-(Di-tert-butyl-phosphanyl)-1′,3′,5′-triphenyl-140 H-[1,4′]bipyrazolyl and 241 mg (4.30 mmol, 2.15 eq) of KOH, was added 3 ml tertamylacohol and 400 μl of water and the suspension is stirred for 10 minutes at 90° C. 521 mg (2.00 mmol, 1 eq) of 3-(2-Amino-[1,2,4]triazolo[1,5-a]pyridin-6-yl)-4-chloro-phenol and 744 mg (3.43 mmol, 1.2 eq) of (3-Bromo-5-hydroxymethyl-phenyl)-methanol are then added, followed by another 3 ml of tertamyl alcohol and 400 μl of water and the mixture is stirred at 90° C. under argon for 10 hours. The compound is extracted by 3 times Ethyl acetate, washed with brine. The organic layers are then dried over Na₂SO₄, filtered and evaporated. The compound is cristallized in methanol/ether and is filtered and washed with ether. It is then purified by preparative HPLC using a ZORBAX, SB-C18 column (21.2 mm×100 mm, 5 μm). The gradient was performed using a H2O/Acetonitrile gradient (from 30% water to 95% acetonitrile) at a flow rate of 50 ml/mn during 15 min to give 70 mg (0.177 mmol, 9%).

(3-Bromo-5-hydroxymethyl-phenyl)-methanol could be synthetically obtained using classical methods of organic synthesis starting from 5-Bromo-isophthalic acid dimethyl ester which has been purchased at Alfa Aesar. Other derivatives could be synthetically obtained using classical methods of organic synthesis.

Synthesis of compound of the Invention N° 19—Method 2

To 49 mg (0.05 mmol, 0.03 eq) of Pd₂(dba)₃, 16 mg (0.03 mmol, 0.02 eq) of 5-(Di-tert-butyl-phosphanyl)-1′,3′,5′-triphenyl-1′H-[1,4′]bipyrazolyl and 241 mg (4.30 mmol, 2.15 eq) of KOH, was added 3 ml tertamylacohol and 400 μl of water and the suspension is stirred for 10 minutes at 90° C. 543 mg (2.00 mmol, 1 eq) of 3-(2-Amino-quinazolin-6-yl)-4-chloro-phenol and 668 mg (2.40 mmol, 1.2eq) of (3-Bromo-5-hydroxymethyl-phenyl)-methanol are then added, followed by another 3 ml of tertamyl alcohol and 400 μl of water and the mixture is stirred at 90° C. under argon for 10 hours. The compound is extracted by 3 times Ethyl acetate, washed with brine. The organic layers are then dried over Na₂SO₄, filtered and evaporated. The compound is cristallized in methanol/ether and is filtered and washed with ether. It is then purified by preparative HPLC using a ZORBAX, SB-C18 column (21.2 mm×100 mm, 5 μm). The gradient was performed using a H2O/Acetonitrile gradient (from 30% water to 95% acetonitrile) at a flow rate of 50 ml/mn during 15 min to give 70 mg (0.122 mmol, 6%).

All compounds could also be purified by prep HPLC. We have used an Agilent 1200 series semi-prep with UV detector monitoring at 254 nm. Compounds were purified on a ZORBAX, SB-C18 column (21.2 mm×100 mm, 5 μm). The gradient was typically performed using a H2O/Acetonitrile gradient (from a range starting from 5 to 50% water to 95% acetonitrile) at a flow rate of 50 ml/mn during 15 min.

Compounds n° 1 to 50 of table 1 were made in a similar way as described above.

### Measurement of Inhibition Constants of the Compounds of the Invention.

The screening and profiling experiments described here were performed using Caliper Life Sciences' proprietary LabChip™ technology. Caliper LC3000 and EZ Reader II instruments are widely used throughout the drug discovery process for assay development, primary screening, selectivity screening, generation of Structure-Activity Relationships (SARs) and Mechanism of Action (MOA) studies. The LabChip™ technology is particularly well suited for enzymatic ‘targets’ such as kinases, proteases, phosphatases, histone deacetylases (HDAC), phosphodiesterases (PDE), and acyl-transferases. The key benefit of the technology is the separation and direct measurement of substrates and products, which allows for higher signal-to-noise ratios and fewer false positive/negative results. This direct measurement also allows for the identification and elimination of enzymatic activities that are not associated with the kinase reaction of interest.

General:

The off-chip incubation mobility-shift kinase assay uses a microfluidic chip to measure the conversion of a fluorescent peptide substrate to a phosphorylated product. The reaction mixture, from a microtiter plate well, is introduced through a capillary sipper onto the chip, where the nonphosphorylated substrate and phosphorylated product are separated by electrophoresis and detected via laser-induced fluorescence. The signature of the fluorescence signal over time reveals the extent of the reaction. The phosphorylated product migrates through the chip faster than the non-phosphorylated substrate, and signals from the two forms of the peptide appear as distinct peaks. Caliper's data analysis software (HTSWA) determines peak heights, from which the ratio of product to the peak sum P/(P+S) and percent (%) conversion is calculated. This value is used to compare compound wells to control wells present on the plate, and thereby determine the % inhibition values for the compound. The formula used to calculate % inhibition is as follows, where C_{100%} is the average % conversion of the 100% activity wells and C_{0%} is the average % conversion of the 0% activity wells:

(1-(%conversionofsample—C_{0%})/(C_{100%}−C_{0%}))*100

Specific:

LC3000 Src Assays

Compounds were dissolved in 100% DMSO and diluted to 25× the final desired screening concentration. Serial dilutions were performed to obtain the concentrations specified for particular studies. One μl of each concentration was transferred, in duplicate, to a 384-well Greiner microtiter plate. Generally, 12 μL of enzyme buffer containing purified kinase (various suppliers), 100 mM HEPES, pH 7.5, 1 mM DTT (Calbiochem, 2333153), 10 mM MgCl₂ (Sigma, M-1028) or 10 mM MnCl₂ (Sigma, M-1787) (assay specific), and 0.002% Brij-35 (Sigma, B4184) was added to each well. Compound and enzyme were allowed to pre-incubate for 15 minutes. 12 μL of peptide/ATP buffer containing 100 mM HEPES, pH 7.5, 1.5 μM fluorescein-labeled peptide (specific to kinase of interest), ATP (at K_{M} apparent, Sigma, A9187), and 0.002% Brij-35 was then added to each well to initiate the reaction. Generally, reactions were incubated for 1-1.5 hours at room temperature to obtain adequate (15-40%) conversion of peptide to phosphorylated product in the linear range of the reaction. Reactions were terminated with the addition of 45 μL of Stop Buffer (containing 20 mM EDTA). Plates were then read on the LabChip 3000 using a 12-sipper LabChip. % conversion values and % inhibition values were obtained as described and IC₅₀ curves of compounds were generated using Graphpad Prism Version 4 or 5.01. A nonlinear curve fit using the sigmoidal dose response—variable slope fit was used to graph IC₅₀ curves and determine IC₅₀ values and hillslopes.

It has been shown that the compounds of the Invention have IC50 against Src kinases of <200 nM. Preferred compounds are those having 1050 against Src kinases of <100 nM.

TABLE 1

| | | | MS | IC50 |
|---|---|---|---|---|
| | | | NMR (200 MHz, | (nM) |
| Examples | Name | Structure | DMSOd6) | h Src |
| compound 1 | 4-Chloro-3-[2- (pyridin-4- ylamino)-[1,2, 4]triazolo[1, 5-a]pyridin-6- yl]-phenol | 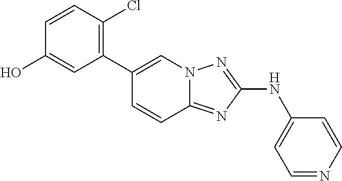 [SMILES: Oc1ccc(Cl)c(-c2ccc3nc(Nc4ccncc4)nn3c2)c1] | M + 1 = 338.1 NMR: 10.28 (s, 1H); 10.00 (bb, 1H); 8.94 (s, 1H); 8.36 (d, 2H); 7.66 (m, 4H); 7.39 (d, 1H); 6.88 (m, 2H) | 73 |
| compound 2 | 4-Chloro-3-[2- (pyridin-3- ylamino)-[1,2, 4]triazolo[1, 5-a]pyridin-6- yl]-phenol | 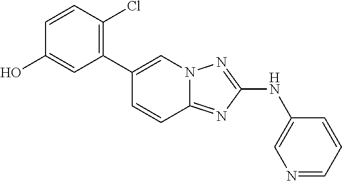 [SMILES: Oc1ccc(Cl)c(-c2ccc3nc(Nc4cccnc4)nn3c2)c1] | M + 1 = 338.1 NMR: 9.87 (s, 1H); 8.80 (s, 2H); 8.16 (d, 1H); 8.09 (d, 1H); 7.63 (s, 2H); 7.35 (m, 2H); 6.88 (m, 2H) | 120 |
| compound 3 | 4-Chloro-3-[2- (pyrimidin-5- ylamino)-[1,2, 4]triazolo[1, 5-a]pyridin-6- yl]-phenol | 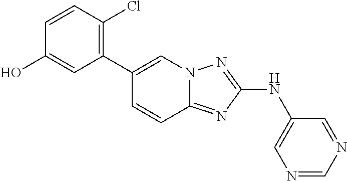 [SMILES: Oc1ccc(Cl)c(-c2ccc3nc(Nc4cncnc4)nn3c2)c1] | M + 1 = 339.1 NMR: 10.17 (s, 1H); 9.96 (s, 1H); 9.15 (s, 2H); 8.95 (s, 1H); 8.75 (s, 1H); 7.67 (s, 2H); 7.39 (d, 1H); 6.91 (m, 2H) | 150 |
| compound 4 | 4-Chloro-3-[2- (5-hydroxy- methyl- pyridin-3- ylamino)-[1,2, 4]triazolo[1, 5-a]pyridin-6- yl]-phenol | 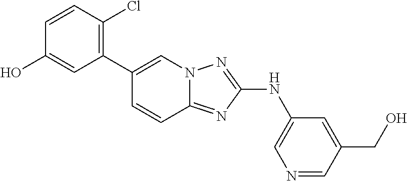 [SMILES: OCc1cncc(Nc2nc3ccc(-c4cc(O)ccc4Cl)cn3n2)c1] | M + 1 = 368.1 NMR: 10.89 (s, 1H); 9.85 (s, 1H); 8.79 (s, 1H); 8.70 (d, 1H); 8.10 (s, 1H); 8.04 (s, 1H); 7.63 (s, 2H); 7.37 (d, 1H); 6.87 (m, 2H); 5.64 (bb, 1H), 4.52 (d, 2H) | 39 |
| compound 5 | 3-[2-(3,5-Bis- hydroxy- methyl- phenylamino)- [1,2,4]triazolo [1,5-a]pyridin- 6-yl]-4-chloro- phenol | 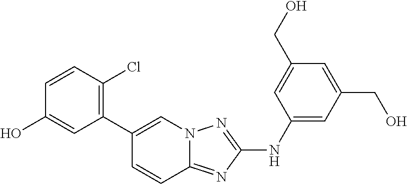 [SMILES: OCc1cc(CO)cc(Nc2nc3ccc(-c4cc(O)ccc4Cl)cn3n2)c1] | M + 1 = 397.1 NMR: 9.94 (s, 1H); 9.60 (s, 1H); 8.84 (s, 1H); 7.61 (s, 2H); 7.54 (s, 2H); 7.39 (d, 1H); 6.87 (m, 3H); 5.15 (t, 2H, OH); 4.47 (d, 4H) | 5 |
| compound 7 | 4-Chloro-3-[2- (6-methoxy- pyridin-3- ylamino)-[1,2, 4]triazolo[1, 5-a]pyridin-6- yl]-phenol | 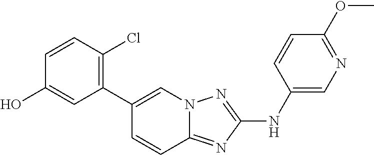 [SMILES: COc1ccc(Nc2nc3ccc(-c4cc(O)ccc4Cl)cn3n2)cn1] | M + 1 = 368.1 NMR: 9.95 (s, 1H); 9.57 (s, 1H); 8.84 (s, 1H); 8.50 (s, 1H); 8.05 (d, 1H); 7.60 (s, 2H); 7.41 (d, 1H); 6.89 (m, 3H); 3.81 (s, 3H) | 80 |
| compound 8 | 4-Chloro-3-{2- [5-(2- pyrrolidin- 1-yl-ethoxy)- pyridin-2- ylamino]-[1,2, 4]triazolo[1, 5-a]pyridin-6- yl}-phenol | 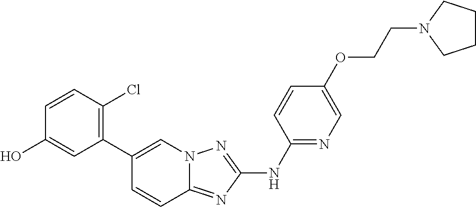 [SMILES: Oc1ccc(Cl)c(-c2ccc3nc(Nc4ccc(OCCN5CCCC5)cn4)nn3c2)c1] | M + 1 = 451.1 NMR: 10.05 (s, 1H); 10.02 (s, 1H); 8.87 (s, 1H); 8.08 (m, 2H); 7.64 (s, 2H); 7.53 (dd, 1H); 7.39 (d, 1H); 6.92 (m, 2H); 4.30 (t, 2H); 3.35 (t, 2H); 3.13 (bb, 4H); 1.88 (bb, 4H) | 100 |
| compound 10 | 4-Chloro-3-(2- {6-[4-(2- hydroxy- ethyl)- piperazin-1- yl]-pyridin-3- ylamino}-[1,2, 4]triazolo[1, 5-a]pyridin-6- yl)-phenol | 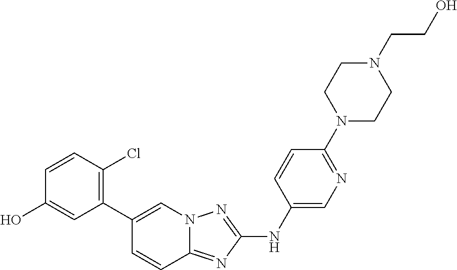 [SMILES: OCCN1CCN(c2ccc(Nc3nc4ccc(-c5cc(O)ccc5Cl)cn4n3)cn2)CC1] | M + 1 = 466.2 NMR: 10.85 (s, 1H); 10.01 (s, 1H); 8.88 (s, 1H); 8.61 (s, 1H); 8.21 (d, 1H); 7.66 (s, 2H); 7.38 (m, 2H); 6.89 (m, 2H); 4.35 (t, 2H); 3.83 (t, 2H); 3.63 (m, 4H); 3.25 (m, 4H) | 14 |
| compound 12 | 4-Chloro-3-[2- (pyridin-2- ylamino)-[1,2, 4]triazolo[1, 5-a]pyridin-6- yl]-phenol | 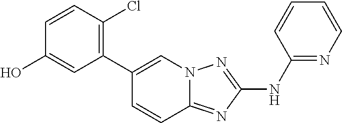 [SMILES: Oc1ccc(Cl)c(-c2ccc3nc(Nc4ccccn4)nn3c2)c1] | M + 1 = 338.0 NMR: 12.42 (s, 1H); 10.17 (bb, 1H); 9.07 (s, 1H); 8.46 (d, 1H); 8.26 (t, 1H); 7.85 (m, 3H); 7.41 (d, 1H); 7.32 (t, 1H); 6.97 (m, 2H) | 170 |
| compound 13 | 4-Chloro-3-[2- (2-hydroxy- methyl- pyridin-4- ylamino)-[1,2, 4]triazolo[1, 5-a]pyridin-6- yl]-phenol | 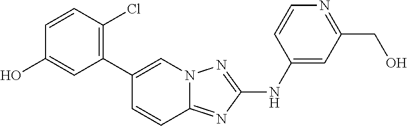 [SMILES: OCc1cc(Nc2nc3ccc(-c4cc(O)ccc4Cl)cn3n2)ccn1] | M + 1 = 368.1 NMR: 10.26 (s, 1H); 8.88 (s, 1H); 8.26 (d, 1H); 7.67 (m, 4H); 7.31 (d, 1H); 6.83 (m, 2H); 4.50 (s, 2H) | 27 |
| compound 14 | 4-Chloro-3-[2- (6-hydroxy- methyl- pyridin-3- ylamino)-[1,2, 4]triazolo[1, 5-a]pyridin-6- yl]-phenol | 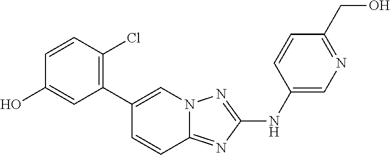 [SMILES: OCc1ccc(Nc2nc3ccc(-c4cc(O)ccc4Cl)cn3n2)cn1] | M + 1 = 368.0 NMR: 9.85 (s, 1H); 8.78 (d, 2H); 8.18 (d, 1H); 7.63 (s, 2H); 7.39 (d, 2H); 6.90 (m, 2H); 5.32 (bb, 1H); 4.50 (s, 2H) | 47 |
| compound 16 | 3-[2-(3,5-Bis- hydroxy- methyl- phenylamino)- quinazolin-6- yl]-4-chloro- phenol | 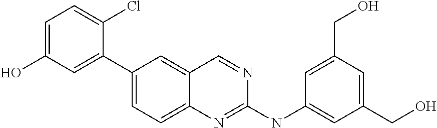 [SMILES: OCc1cc(CO)cc(Nc2ncc3cc(-c4cc(O)ccc4Cl)ccc3n2)c1] | M + 1 = 408.0 NMR: 10.0 (bb, 1H, OH); 9.91 (s, 1H); 9.35 (s, 1H); 7.95- 7.83 (m, 4H); 7.69 (d, 1H); 7.37 (d, 1H); 6.94 (s, 1H); 6.84 (m, 2H); 5.18 (bb, 2H); 4.51 (s, 4H) | 0.1 |
| Compound 17 | 4-Chloro-3-[2- (pyridin-3- ylamino)- quinazolin-6- yl]-phenol | 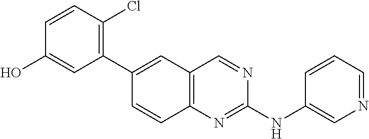 [SMILES: Oc1ccc(Cl)c(-c2ccc3nc(Nc4cccnc4)ncc3c2)c1] | M + 1 = 349.0 NMR: 10.18 (s, 1H); 9.41 (s, 1H); 9.10 (s, 1H); 8.47 (d, 1H); 8.21 (d, 1H); 7.99 (s, 1H); 7.88 (dd, 1H); 7.75 (d, 1H); 7.38 (m, 2H); 6.84 (m, 2H) | 2 |
| Compound 18 | 4-Chloro-3-[2- (1H-indol-6- ylamino)-[1,2, 4]triazolo[1, 5-a]pyridin-6- yl]-phenol | 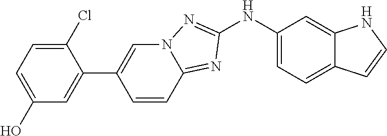 [SMILES: Oc1ccc(Cl)c(-c2ccc3nc(Nc4ccc5cc[nH]c5c4)nn3c2)c1] | M + 1 = 376.1 NMR: 10.93 (s, 1H); 9.48 (s, 1H); 8.79 (s, 1H); 7.99 (s, 1H); 7.58 (s, 2H); 7.39 (m, 2H); 7.16 (m, 2H); 6.89 (m, 2H); 6.31 (s, 1H) | 39 |
| Compound 19 | 4-[6-(2-Chloro- 5-hydroxy- phenyl)-[1,2, 4]triazolo[1, 5-a]pyridin-2- ylamino]- pyridin-2-ol | 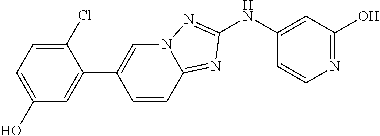 [SMILES: Oc1ccc(Cl)c(-c2ccc3nc(Nc4ccnc(O)c4)nn3c2)c1] | M + 1 = 354.1 NMR: 10.93 (s, 1H); 10.06 (s, 1H); 9.97 (s, 1H); 9.94 (s, 1H); 7.68 (s, 2H); 7.39 (s, 1H); 7.24 (s, 1H); 6.89 (m, 3H); 6.35 (d, 1H) | 28 |
| Compound 20 | 4-Chloro-3-[2- (2-methoxy- pyridin-4- ylamino)-[1,2, 4]triazolo[1, 5-a]pyridin-6- yl]-phenol | 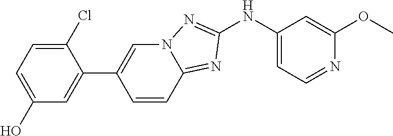 [SMILES: COc1cc(Nc2nc3ccc(-c4cc(O)ccc4Cl)cn3n2)ccn1] | M + 1 = 368.1 NMR: 11.11 (s, 1H); 10.03 (s, 1H); 9.02 (s, 1H); 8.10 (d, 1H); 7.76 (m, 2H); 7.58 (s, 1H); 7.40 (m, 2H); 6.93 (m, 2H); 4.01 (s, 3H) | 6 |
| Compound 21 | 4-Chloro-3-[2- (5-hydroxy- methyl- pyridin-3- ylamino)- quinazolin-6- yl]-phenol | 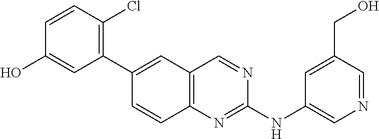 [SMILES: OCc1cncc(Nc2ncc3cc(-c4cc(O)ccc4Cl)ccc3n2)c1] | M + 1 = 379.0 NMR: 10.17 (s, 1H); 9.94 (bb, 1H); 9.41 (s, 1H); 9.02 (s, 1H); 8.41 (s, 1H); 8.16 (s, 1H); 7.99 (d, 1H); 7.89 (dd, 1H); 7.75 (d, 1H); 7.38 (d, 1H); 6.85 (m, 2H); 5.37 (bb, 1H); 4.57 (s, 2H) | 0.4 |
| Compound 25 | 4-Chloro-3- {2-[6-(2- pyrrolidin- 1-yl-ethoxy)- pyridin-3- ylamino]-[1,2, 4]triazolo[1, 5-a]pyridin-6- yl}-phenol | 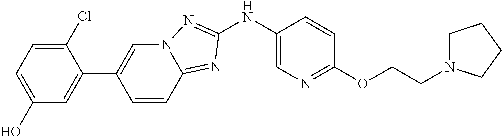 [SMILES: Oc1ccc(Cl)c(-c2ccc3nc(Nc4ccc(OCCN5CCCC5)nc4)nn3c2)c1] | M + 1 = 451.1 NMR : 9.98 (bb, 1H); 9.58 (s, 1H); 8.83 (s, 1H); 8.49 (s, 1H); 8.04 (d, 1H); 7.60 (s, 2H); 7.38 (d, 1H); 6.84 (m, 3H); 4.33 (t, 2H); 2.89 (t, 2H); 2.64 (m, 4H); 1.72 (m, 4H) | 23 |
| Compound 26 | 4-Chloro-3- {2-[5-(2- pyrrolidin- 1-yl-ethoxy)- pyridin-3- ylamino]-[1,2, 4]triazolo[1, 5-a]pyridin-6- yl}-phenol | 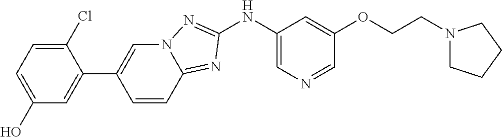 [SMILES: Oc1ccc(Cl)c(-c2ccc3nc(Nc4cncc(OCCN5CCCC5)c4)nn3c2)c1] | M + 1 = 451.2 NMR: 9.97 (s, 1H); 8.91 (s, 1H); 8.44 (s, 1H); 7.88 (m, 2H); 7.64 (s, 2H); 7.37 (d, 1H); 6.89 (m, 2H); 4.14 (t, 2H); 2.81 (t, 2H); 2.55 (m, 4H); 1.68 (m, 4H) | 14 |
| Compound 27 | 4-Chloro-3-(2- {6-[4-(2- hydroxy- ethyl)- piperazin-1- yl]-2-methyl- pyrimidin-4- ylamino}-[1,2, 4]triazolo[1, 5-a]pyridin-6- yl)-phenol | 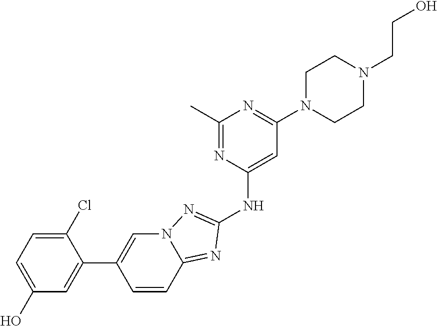 [SMILES: Cc1nc(Nc2nc3ccc(-c4cc(O)ccc4Cl)cn3n2)cc(N2CCN(CCO)CC2)n1] | M + 1 = 481.2 NMR: 10.35 (bb, 1H); 10.17 (s, 1H); 8.93 (s, 1H); 7.67 (s, 2H); 7.37 (m, 2H); 6.93 (m, 2H); 4.50 (bb, 1H); 3.56 (m, 6H); 2.48 (m, 4H); 2.42 (t, 2H); 2.31 (s, 3H) | 83 |
| Compound 28 | 4-Chloro-3-(2- {3-[4-(2- hydroxy- ethyl)- piperazin-1- yl]-5-methyl- phenylamino}- [1,2,4]triazolo [1,5-a]pyridin- 6-yl)-phenol | 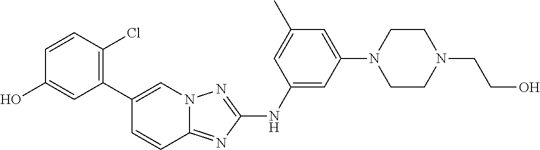 [SMILES: Cc1cc(Nc2nc3ccc(-c4cc(O)ccc4Cl)cn3n2)cc(N2CCN(CCO)CC2)c1] | M + 1 = 479.1 NMR: 9.88 (bb, 1H); 9.35 (s, 1H); 8.81 (s, 1H); 7.55 (s, 2H); 7.34 (d, 1H); 7.12 (s, 1H); 6.95 (s, 1H); 6.84 (m, 2H); 6.27 (s, 1H); 4.57 (t, 1H); 3.49 (q, 2H); 3.07 (m, 4H); 2.52 (m, 4H); 2.39 (t, 2H); 2.19 (s, 3H) | 3 |
| Compound 29 | 4-Chloro-3-[2- (3,4,5- trimethoxy- phenylamino)- [1,2,4]triazolo [1,5-a]pyridin- 6-yl]-phenol | 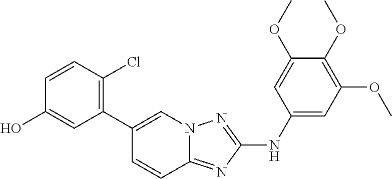 [SMILES: COc1cc(Nc2nc3ccc(-c4cc(O)ccc4Cl)cn3n2)cc(OC)c1OC] | M + 1 = 427.1 NMR: 9.92 (s, 1H); 9.51 (s, 1H); 8.86 (s, 1H); 7.60 (s, 2H); 7.39 (d, 1H); 7.12 (s, 2H); 6.91 (m, 2H); 3.78 (s, 6H); 3.61 (s, 3H) | 2 |
| Compound 30 | 4-Chloro-3-{2- [3-(2-hydroxy- ethyl)-3H- benzoimidazol- 5-ylamino]- [1,2,4]triazolo [1,5-a]pyridin- 6-yl}-phenol | 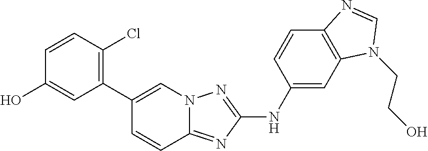 [SMILES: OCCn1cnc2ccc(Nc3nc4ccc(-c5cc(O)ccc5Cl)cn4n3)cc21] | M + 1 = 421.2 NMR: 10.21 (bb, 1H, OH); 9.68 (s, 1H); 8.86 (s, 1H); 8.04 (s, 1H); 8.03 (s, 1H); 7.60 (s, 2H); 7.53 (dd, 1H); 7.44 (d, 1H); 7.38 (d, 1H); 6.93 (s, 1H); 6.86 (d, 1H); 4.69 (bb, 1H); 4.23 (m, 2H); 3.80 (m, 2H) | 7 |
| Compound 31 | 4-Chloro-3-[2- (pyridin-3- ylamino)-[1,2, 4]triazolo[1, 5-a]pyridin-7- yl]-phenol | 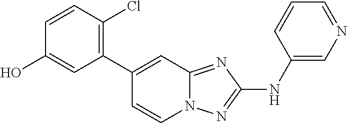 [SMILES: Oc1ccc(Cl)c(-c2ccn3nc(Nc4cccnc4)nc3c2)c1] | M + 1 = 337.9 NMR: 10.11 (bb, 1H); 9.90 (s, 1H); 8.84 (m, 2H); 8.19 (d, 1H); 8.12 (d, 1H); 7.60 (s, 1H); 7.36 (m, 2H); 7.08 (dd, 1H); 6.88 (m, 2H) | 69 |
| Compound 33 | 4-Chloro-3- {2-[2-(2- pyrrolidin- 1-yl-ethoxy)- pyridin-4- ylamino]- [1,2,4]triazolo [1,5-a]pyridin- 6-yl}-phenol | 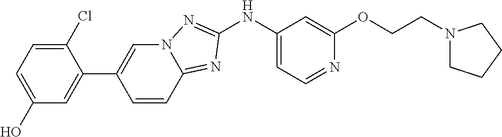 [SMILES: Oc1ccc(Cl)c(-c2ccc3nc(Nc4ccnc(OCCN5CCCC5)c4)nn3c2)c1] | M + 1 = 451.2 NMR: 10.10 (bb, 1H); 9.21 (s, 1H); 8.73 (s, 1H); 7.88 (s, 1H); 7.54 (s, 2H); 7.46 (s, 1H); 7.37 (d, 1H); 6.89 (m, 2H); 4.17 t, 2H); 3.2 (m, 4H); 2.86 (m, 4H); 2.71 (t, 2H) | 29 |
| Compound 34 | 3-[2-(3,5-Bis- hydroxy- methyl- phenylamino)- [1,2,4]triazolo [1,5-a]pyridin- 7-yl]-4-chloro- phenol | 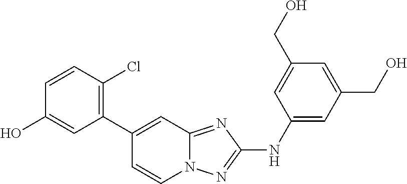 [SMILES: OCc1cc(CO)cc(Nc2nc3cc(-c4cc(O)ccc4Cl)ccn3n2)c1] | M + 1 = 297.0 NMR: 10.01 (bb, 1H); 9.58 (s, 1H); 8.81 (d, 1H); 7.54 (m, 3H); 7.39 (d, 1H); 7.03 (dd, 1H); 6.86 (m, 3H); 5.15 (t, 2H); 4.47 (d, 4H) | 9 |
| Compound 35 | 3-[2-(3,4-Bis- hydroxy- methyl- phenylamino)- [1,2,4]triazolo [1,5-a]pyridin- 6-yl]-4-chloro- phenol | 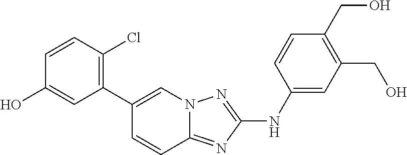 [SMILES: OCc1ccc(Nc2nc3ccc(-c4cc(O)ccc4Cl)cn3n2)cc1CO] | M + 1 = 397.0 NMR: 9.96 (bb, 1H); 9.60 (s, 1H); 8.84 (s, 1H); 7.61 (m, 4H); 7.39 (d, 1H); 7.25 (d, 1H); 6.90 (m, 2H); 5.09 (t, 1H); 4.93 (t, 1H); 4.54 (d, 2H); 4.47 (d, 2H) | 34 |
| Compound 36 | 4-Chloro-3-[2- (3,4,5- trimethoxy- phenylamino)- quinazolin-6- yl]-phenol | 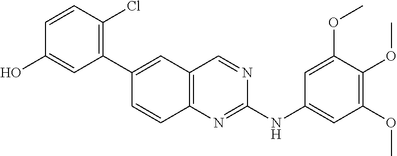 [SMILES: COc1cc(Nc2ncc3cc(-c4cc(O)ccc4Cl)ccc3n2)cc(OC)c1OC] | M + 1 = 438.0 NMR: 9.90 (bb, 1H); 9.86 (s, 1H); 9.34 (s, 1H); 7.94 (d, 1H); 7.86 (dd, 1H); 7.71 (d, 1H); 7.50 (s, 2H); 7.38 (d, 1H); 6.87 (m, 2H); 3.82 (s, 6H); 3.64 (s, 3H) | 0.5 |
| Compound 38 | 4-Chloro-3-(2- {2-[4-(2- hydroxy- ethyl)- piperazin-1- yl]-pyridin-4- ylamino}-[1,2, 4]triazolo[1, 5-a]pyridin-6- yl)-phenol | 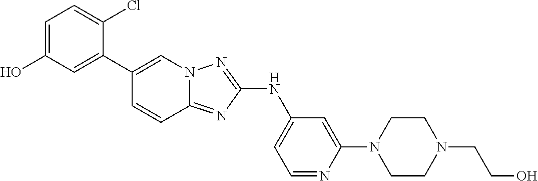 [SMILES: OCCN1CCN(c2cc(Nc3nc4ccc(-c5cc(O)ccc5Cl)cn4n3)ccn2)CC1] | M + 1 = 466.0 NMR: 9.93 (bb, 2H, NH, OH); 8.92 (s, 1H); 7.92 (d, 1H); 7.65 (m, 2H); 7.39 (d, 1H); 7.19 (s, 1H); 6.99 (d, 1H); 6.92 (d, 1H); 6.87 (dd, 1H); 4.44 (t, 1H, OH); 3.54 (q, 2H); 3.43 (m, 4H); 2.52 (m, 4H); 2.44 (t, 2H) | 13 |
| Compound 39 | 4-Chloro-3-{7- methoxy-2-[4- (2-pyrrolidin- 1-yl-ethoxy)- phenylamino]- [1,2,4]triazolo [1,5-a]pyridin- 6-yl}-phenol | 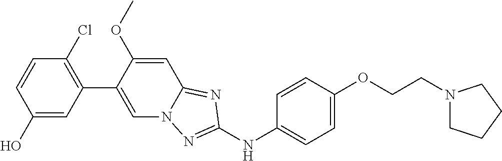 [SMILES: COc1cc2nc(Nc3ccc(OCCN4CCCC4)cc3)nn2cc1-c1cc(O)ccc1Cl] | M + 1 = 480.2 NMR: 9.83 (s, 1H); 9.29 (s, 1H); 8.55 (s, 1H); 7.58 (d, 2H); 7.31 (d, 1H); 7.06 (s, 1H); 6.84 (m, 4H); 4.01 (t, 2H); 3.84 (s, 3H); 2.78 (t, 2H); 2.55 (m, 4H); 1.69 (m, 4H) | 69 |
| Compound 40 | 4-Chloro-3-[2- (6-methoxy- pyridin-3- ylamino)- quinazolin-6- yl]-phenol | 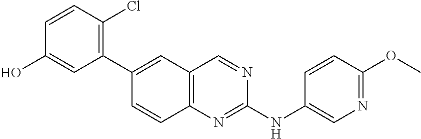 [SMILES: COc1ccc(Nc2ncc3cc(-c4cc(O)ccc4Cl)ccc3n2)cn1] | M + 1 = 379.0 NMR: 9.91 (s, 1H); 9.90 (bb, 1H); 9.34 (s, 1H); 8.80 (d, 1H); 8.21 (dd, 1H); 7.95 (d, 1H); 7.85 (dd, 1H); 7.78 (d, 1H); 7.38 (d, 1H); 6.85 (m, 3H); 3.84 (s, 3H) | 10 |
| Compound 41 | 4-Chloro-3-(2- {4-[2-(1-oxy- pyrrolidin-1- yl)-ethoxy]- phenylamino}- [1,2,4]triazolo [1,5-a]pyridin- 6-yl)-phenol | 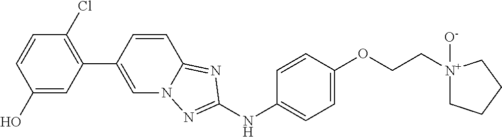 [SMILES: [O-][N+]1(CCOc2ccc(Nc3nc4ccc(-c5cc(O)ccc5Cl)cn4n3)cc2)CCCC1] | M + 1 = 466.2 NMR: 11.07 (bb, 1H); 9.44 (s, 1H); 8.78 (s, 1H); 7.60 (d, 2H); 7.55 (s, 2H); 7.33 (d, 1H); 6.89 (m, 3H); 6.82 (dd, 1H); 4.48 (t, 2H); 3.68 (t, 2H); 3.47 (m, 4H); 2.16 (m, 2H); 1.93 (m, 2H) | 55 |
| Compound 42 | 4-Chloro-3-[2- (1H-indol-6- ylamino)- quinazolin-6- yl]-phenol | 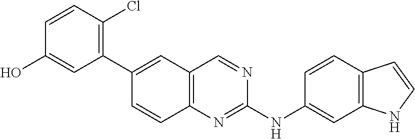 [SMILES: Oc1ccc(Cl)c(-c2ccc3nc(Nc4ccc5cc[nH]c5c4)ncc3c2)c1] | M + 1 = 387.0 NMR: 11.02 (s, 1H); 9.99 (bb, 1H); 9.87 (s, 1H); 9.313 (s, 1H); 8.37 (s, 1H); 7.94 (d, 1H); 7.83 (dd, 1H); 7.66 (d, 1H); 7.40 (m, 3H); 7.25 (t, 1H); 6.85 (m, 2H); 6.36 (s, 1H) | 100 |
| Compound 43 | 4-Chloro-3-[2- (2-hydroxy- methyl- pyridin-4- ylamino)- quinazolin-6- yl]-phenol | 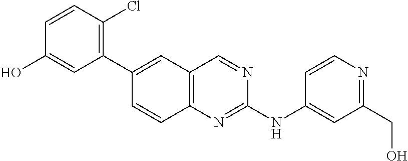 [SMILES: OCc1cc(Nc2ncc3cc(-c4cc(O)ccc4Cl)ccc3n2)ccn1] | M + 1 = 379.1 NMR: 10.39 (s, 1H); 9.97 (bb, 1H); 9.46 (s, 1H); 8.33 (d, 2H); 8.04 (dd, 1H); 7.94 (m, 2H); 7.81 (d, 1H); 7.38 (d, 1H); 6.90 (d, 1H); 6.85 (dd, 1H); 5.36 (bb, 1H, OH); 4.54 (s, 2H) | 0.7 |
| Compound 44 | 1-(2-{5-[6-(2- Chloro-5- hydroxy- phenyl)-[1,2, 4]triazolo[1, 5-a]pyridin-2- ylamino]- pyridin-2- yloxy}-ethyl)- pyrrolidin-2- one | 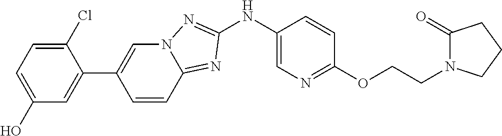 [SMILES: O=C1CCCN1CCOc1ccc(Nc2nc3ccc(-c4cc(O)ccc4Cl)cn3n2)cn1] | M + 1 = 466.1 NMR: 9.92 (s, 1H); 9.55 (s, 1H); 8.83 (s, 1H); 8.49 (s, 1H); 8.04 (d, 1H); 7.60 (s, 2H); 7.38 (d, 1H); 6.90 (s, 1H); 6.86 (d, 1H); 6.80 (d, 1H); 4.32 (t, 2H); 3.53 (t, 2H); 3.47 (t, 2H); 2.20 (t, 2H); 1.90 (quint, 2H) | 57 |
| Compound 45 | 4-Chloro-3-{2- [1-(2-hydroxy- ethyl)-1H- benzimidazol- 5-ylamino]- quinazolin-6- yl}-phenol | 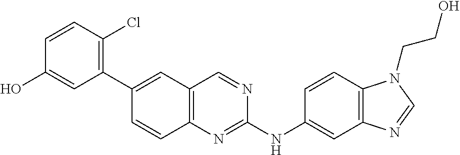 [SMILES: OCCn1cnc2cc(Nc3ncc4cc(-c5cc(O)ccc5Cl)ccc4n3)ccc21] | M + 1 = 432.1 NMR: 9.89 (s, 1H); 9.88 (bb, 1H); 9.33 (s, 1H); 8.48 (s, 1H); 8.12 (s, 1H); 7.94 (s, 1H); 7.83 (d, 1H); 7.68 (m, 2H); 7.54 (d, 1H); 7.38 (d, 1H); 6.89 (d, 1H); 6.84 (dd, 1H); 4.99 (bb, 1H, OH); 4.27 (t, 2H); 3.75 (m, 2H) | 0.5 |
| Compound 46 | 4-Chloro-3-(2- {3-[4-(2- hydroxy- ethyl)- piperazin-1- yl]-5-methyl- phenylamino}- quinazolin-6- yl)-phenol | 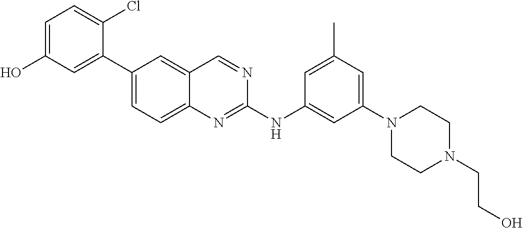 [SMILES: Cc1cc(Nc2ncc3cc(-c4cc(O)ccc4Cl)ccc3n2)cc(N2CCN(CCO)CC2)c1] | M + 1 = 490.2 NMR: 9.19 (s, 1H); 8.46 (s, 1H); 7.84 (m, 2H); 7.70 (d, 1H); 7.62 (s, 1H); 7.32 (d, 1H); 7.15 (s, 1H); 6.88 (d, 1H); 6.81 (dd, 1H); 6.56 (s, 1H); 3.85 (t, 2H); 3.41 (m, 4H); 3.16 (m, 4H); 3.00 (t, 2H); 2.34 (s, 3H) | 0.8 |
| Compound 47 | 4-Chloro-3-(2- {3-[4-(2- hydroxy- ethyl)- piperazin-1- yl]-5-hydroxy- methyl- phenylamino}- [1,2,4]triazolo [1,5-a]pyridin- 6-yl)-phenol | 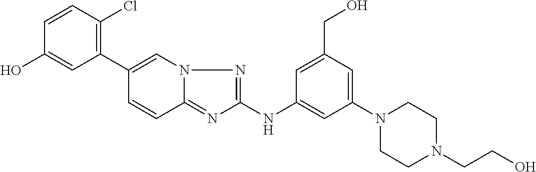 [SMILES: OCCN1CCN(c2cc(CO)cc(Nc3nc4ccc(-c5cc(O)ccc5Cl)cn4n3)c2)CC1] | M + 1 = 495.1 NMR: 9.43 (s, 1H); 8.85 (s, 1H); 8.18 (s, 1H); 7.59 (s, 2H); 7.38 (d, 1H); 7.27 (s, 1H), 7.10 (s, 1H); 6.91 (d, 1H); 6.86 (dd, 1H); 6.46 (s, 1H); 4.41 (s, 2H); 3.54 (t, 2H); 3.13 (m, 4H); 2.58 (m, 4H); 2.45 (t, 2H) | 1 |
| Compound 48 | Benzoic acid 4- chloro-3-(2-{3- [4-(2-hydroxy- ethyl)- piperazin-1- yl]-5-methyl- phenylamino}- [1,2,4]triazolo [1,5-a]pyridin- 6-yl)-phenyl ester | 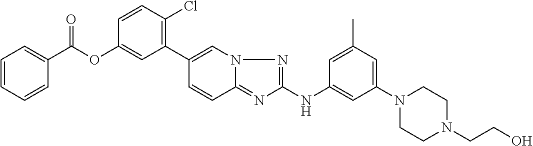 [SMILES: Cc1cc(Nc2nc3ccc(-c4cc(OC(=O)c5ccccc5)ccc4Cl)cn3n2)cc(N2CCN(CCO)CC2)c1] | M + 1 = 583.1 NMR: 9.41 (s, 1H); 8.94 (s, 1H); 8.15 (d, 2H); 7.80-7.61 (m, 7H); 7.46 (dd, 1H); 7.16 (s, 1H); 7.00 (s, 1H); 6.32 (s, 1H); 4.54 (bb, 1H); 3.54 (q, 2H); 3.11 (m, 4H); 2.57 (m, 4H); 2.45 (t, 2H); 2.22 (s, 3H) | NA (not appli- cable) |
| Compound 49 | Benzoic acid 4- chloro-3-(2-{3- [4-(2-hydroxy- ethyl)- piperazin-1-yl]- 5-methyl- phenylamino}- quinazolin-6- yl)-phenyl ester | 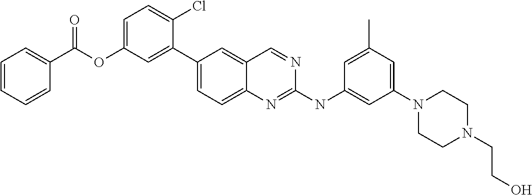 [SMILES: Cc1cc(Nc2ncc3cc(-c4cc(OC(=O)c5ccccc5)ccc4Cl)ccc3n2)cc(N2CCN(CCO)CC2)c1] | M + 1 = 594.1 NMR: 9.74 (s, 1H); 9.34 (s, 1H); 8.15 (d, 2H); 8.03 (d, 1H); 7.93 (dd, 1H); 7.73 (m, 3H); 7.62 (m, 3H); 7.55 (d, 1H); 7.43 (dd, 1H); 7.18 (s, 1H); 6.43 (s, 1H); 4.45 (bb, 1H); 3.55 (q, 2H); 3.15 (m, 4H); 2.58 (m, 4H); 2.45 (t, 2H); 2.26 (s, 3H) | 11 |
| Compound 50 | 4-Chloro-3-(2- {3-[4-(2- hydroxy- ethyl)- piperazin-1- yl]-5-hydroxy- methyl- phenylamino}- quinazolin-6- yl)-phenol | 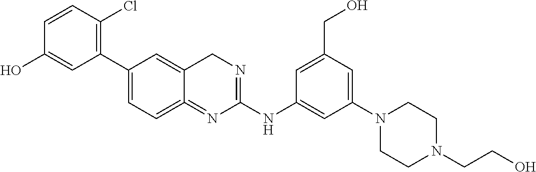 | M + 1 = 506.01 NMR: 9.77 (s, 1H); 9.33 (s, 1H); 8.16 (s, 1H); 7.94 (s, 1H); 7.86 (dd, 1H); 7.71 (s, 1H); 7.67 (d, 1H); 7.38 (d, 1H), 7.31 (s, 1H); 6.88 (s, 1H); 6.86 (dd, 1H); 6.58 (s, 1H); 4.44 (s, 2H); 3.56 (t, 2H); 3.17 (m, 4H); 2.61 (m, 4H); 2.47 (t, 2H) | 0.2 |

All compounds of the invention are white or pale yellow powders, and in solution become pale yellow or colourless when in solution at the maximum concentration of solubilisation at pH 5.

Other data regarding some compounds of the invention are as follows:

TABLE 2

| | | | | | Flux between |
|---|---|---|---|---|---|
| | | | | | 2 and 4 hours |
| | In vitro | | | Solubility | through 0.5 |
| | potency—IC50 | | | HPbCD 7% | cm2 of rabbit |
| | (nM) Src/ | | | pH 5 (mg/ml) | cornea |
| Compound | Lyn (h) | | Colour | measured | (μg/h/cm2) |
| Compound | <1 | nM | Pale | 1.2 | 17 |
| 16 | | | Yellow | | |
| Compound | 0.7 | nM | Pale | 1.5 | 6 |
| 43 | | | Yellow | | |
| Compound | 1 | nM | Pale | 2.3 | 8 |
| 45 | | | Yellow | | |
| Compound | 0.8 | nM | Pale | >10 | 13 |
| 46 | | | Yellow | | |
| Compound | 5 | nM | White | 1.3 | 13 |
| 5 | | | | | |
| Compound | 14 | nM | White | 5.69 | 30 |
| 10 | | | | | |
| Compound | 27 | nM | White | 2.38 | 13 |
| 13 | | | | | |
| Compound | 23 | nM | White | 6.5 | 14 |
| 25 | | | | | |
| Compound | 83 | nM | White | >10 | 19 |
| 27 | | | | | |
| Compound | 3 | nM | White | 7.5 | 8 |
| 28 | | | | | |

Compounds n° 1 to 50, including prodrugs (compounds n° 48 and 49), not listed in table 2 show similar solubility and Corneal flux.

Accordingly above-recited problems of insolubility and coloration of compounds of the prior art have been solved by the compounds of the current invention. Compounds of the invention are both colourless and readily soluble in aqueous formulations suitable for delivery by eye drops.

Experimental—Ussing Chamber

Ussing chambers were used for the permeation study each day of experiment.

3 mL of solution were placed in donor side of Ussing chambers and 3 mL of Ringer solution were in receiver side. Freshly removed rabbit corneal tissue were placed between the two half chambers.

Temperature was maintained at 37° C. during all the flux study and oxygenation was provided by a continuous perfusion of carbogen (oxygen/carbonic acid) (95/5),

Rabbits were euthanized and the 6 corneas were removed and used immediately. 500 μL of receptor side liquid were removed from Ussing chambers and replaced by fresh buffer. The samples were analyzed immediately (less than 10 hours after collection).

100 μL of donor side liquid were removed from Ussing chambers and not replaced. The samples were diluted immediately (less than 1 hour) after collection and analyzed less than 10 hours after dilution. Analysis was performed on HPLC (Stationary phase: C18 (Particle size: 3 μm Length: 5 cm, using a gradient from 5 to 95% ACN/water (0.1% formic acid)).

At the end of the sampling period, all corneas were discarded.

According to this protocol, it has been demonstrated that compounds of the current invention in these aqueous formulations readily cross the cornea, thus making them suitable for treatment of ophthalmic indications.

Inhibition of Neovascularization in a Rat Model of Laser-Induced Choroidal Neovascularization (CNV)

We investigated the efficacy of topical administration of compound 25 of the invention in reducing choroidal neovascularisation in the rat (Brown Norway, 8 weeks of age).

On day 1, CNV was performed by laser photocoagulation-induced rupture of Bruch's membrane as previously described (Edelman and Casto 2000). An Argon green laser irradiation was delivered through the slit lamp for induce photocoagulation. In each eye, 6-7 focal laser spots were applied concentrically approximately two optic discs from the center. Immediately afterwards, rats were treated with topical solution 6 mg/mL (10 μL) two times daily until sacrifice. 14 days after laser induction of CNV blood vessels were visualized on retinal pigment epithelium-choroid-sclera flat-mount by immunostaining with isolectin B4.

Assessment of CNV response to treatment was performed after capture and area measurement of immunostained vessels representing CNV at the site of laser burn.

Pixel area of vascular budding was traced by 2 trained masked investigators and converted to μm².

### RESULTS

It has been found that compound 25 of invention reduced CNV by 15% compared to control providing evidence that the compounds of the invention are useful to reduce choroidal neovascularization associated with wet age-related macular degeneration.

1.-22. (canceled)

23. A compound of formula (I): 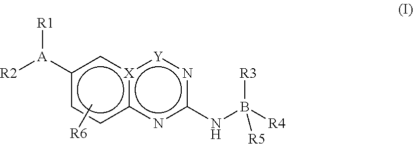
wherein
A is phenyl,
B is phenyl, pyridine, or pyrimidine
R1 and R2 represent independently from each other:
—H,
—OH,
a halogen atom,
with the provisio that R1 and R2 are not simultaneously hydrogen;
R3, R4 and R5 are, independently from each other,
—H,
—(CH₂)ₙOH,
—O(C₁C₆)alkyl,
—(CH₂)ₙ—CO-heterocycloalkyl,
—OH,
-heterocycloalkyl-(CH₂)ₙ—OH,
—(C₁-C₆)alkyl,
—(CH₂)ₙ-heterocycloalkyl,
—(CH₂)ₙ-heterocycloalkyl-(CH₂)ₙ—OH,
—O—(CH₂)ₙ-heterocycloalkyl,
N-oxide wherein the nitrogen atom belongs to B,
—O—(CH₂)ₙ—CO-heterocycloalkyl,
—O—(CH₂)ₙ—OH,
—O(C₁C₆)alkyl-NR7R8,
—(C₁C₆)alkyl-NR7R8,
R3 and R4 form together with B a fused bicycle optionally substituted by R5 with the provisio that when B is phenyl, at least two of R3, R4 and R5 are not hydrogen;
R6 is H, —O(C₁C₆)alkyl, or (C₁C₆)alkyl;
R7 and R8 are independently from each other H or an optionally substituted (C₁C₆)alkyl optionally forming a cycloalkyl;
n is 1, 2 or 3;
X is N or C; and
Y is CH or a covalent bond,
or a prodrug thereof.

24. The compound according to claim 23, wherein said compound has the formula 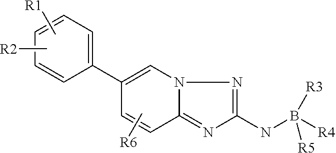
wherein B, R1, R2, R3, R4, R5, R6, R7, and R8 are as defined in claim 23.

25. The compound according to claim 23, wherein said compound has the formula 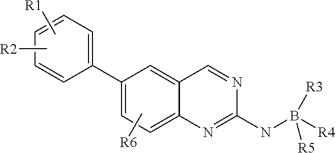
wherein B, R1, R2, R3, R4, R5, R6, R7, and R8 are as defined in claim 23.

26. The compound according to claim 23, wherein said compound has the formula 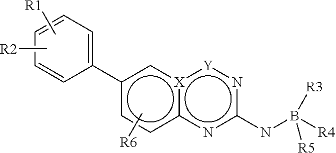
wherein B, X, Y, R1, R2, R3, R4, R5, R6, R7, and R8 are as defined in claim 23.

27. The compound according to claim 23, wherein said compound has the formula 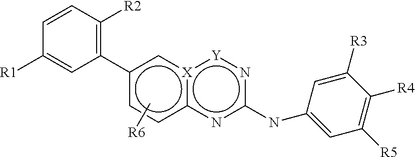
wherein X, Y, R1, R2, R3, R4, R5, R6, R7, and R8 are as defined in claim 23.

28. The compound according to claim 23, wherein said compound has the formula 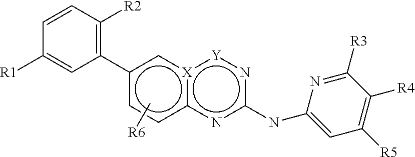
wherein X, Y, R1, R2, R3, R4, R5, R6, R7, and R8 are as defined in claim 23.

29. The compound according to claim 23, wherein said compound has the formula 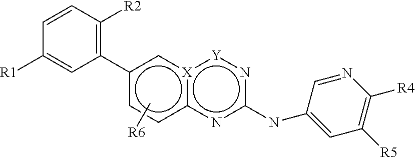
wherein X, Y, R1, R2, R3, R4, R5, R6, R7, and R8 are as defined in claim 23.

30. The compound according to claim 23, wherein said compound has the formula 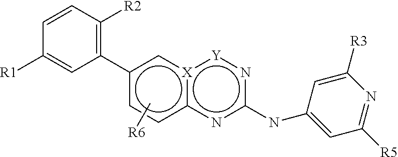
wherein X, Y, R1, R2, R3, R4, R5, R6, R7, and R8 are as defined in claim 23.

31. The compound according to claim 23, wherein said compound has the formula 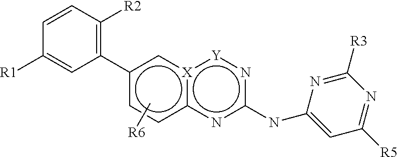
wherein X, Y, R1, R2, R3, R4, R5, R6, R7, and R8 are as defined in claim 23.

32. The compound according to claim 23, wherein said compound has the formula 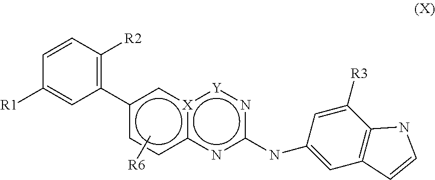
wherein X, Y, R1, R2, R3, R4, R5, R6, R7, and R8 are as defined in claim 23.

33. The compound according to claim 23, wherein said compound has the formula 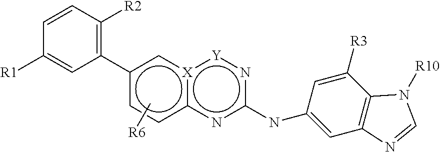
wherein X, Y, R1, R2, R3, R4, R5, R6, R7, and R8 are as defined in claim 23, and wherein R10 is
—H,
—(CH₂)ₙOH,
—O(C₁C₆)alkyl,
—(CH₂)ₙ—CO-heterocycloalkyl,
—OH,
-heterocycloalkyl-(CH₂)ₙ—OH,
-(C₁-C₆)alkyl,
—(CH₂)ₙ-heterocycloalkyl,
—(CH₂)ₙ-heterocycloalkyl-(CH₂)ₙ—OH,
—O—(CH₂)ₙ-heterocycloalkyl,
N-oxide wherein the nitrogen atom belongs to B,
—O—(CH₂)ₙ—CO-heterocycloalkyl,
—O—(CH₂)ₙ—OH,
—O(C₁C₆)alkyl-NR7R8, or
—(C₁C₆)alkyl-NR7R8.

34. The compound according to claim 23, wherein R1 is OH and R2 is a halogen atom.

35. The compound according to claim 23, wherein R3, R4 and R5 represent independently from each other O-alkyl or hydroxyalkyl.

36. The compound according to claim 23, wherein R3, R4 and R5 represent independently from each other —CH₂OH and —O—CH₂—CH₂-heterocycloalkyl.

37. The compound according to claim 23, wherein X represents a carbon atom and Y represents CH, or wherein X represents a nitrogen, and Y represents a bond.

38. The compound according to claim 23, wherein R6 represents a hydrogen atom or CH₃.

39. The compound according to claim 23, wherein said compound is selected from the group consisting of:
4-Chloro-3-[2-(pyridin-4-ylamino)-[1,2,4]triazolo[1,5-a]pyridin-6-yl]-phenol;
4-Chloro-3-[2-(pyridin-3-ylamino)-[1,2,4]triazolo[1,5-a]pyridin-6-yl]-phenol;
4-Chloro-3-[2-(pyrimidin-5-ylamino)-[1,2,4]triazolo[1,5-a]pyridin-6-yl]-phenol;
4-Chloro-3-[2-(5-hydroxymethyl-pyridin-3-ylamino)-[1,2,4]triazolo[1,5-a]pyridin-6-yl]-phenol;
3-[2-(3,5-Bis-hydroxymethyl-phenylamino)-[1,2,4]triazolo[1,5-a]pyridin-6-yl]-4-chloro-phenol;
4-Chloro-3-[2-(6-methoxy-pyridin-3-ylamino)-[1,2,4]triazolo[1,5-a]pyridin-6-yl]-phenol;
4-Chloro-3-{2-[5-(2-pyrrolidin-1-yl-ethoxy)-pyridin-2-ylamino]-[1,2,4]triazolo[1,5-a]pyridin-6-yl}-phenol;
4-Chloro-3-(2-{6-[4-(2-hydroxy-ethyl)-piperazin-1-yl]-pyridin-3-ylamino}-[1,2,4]triazolo[1,5-a]pyridin-6-yl)-phenol;
4-Chloro-3-[2-(pyridin-2-ylamino)-[1,2,4]triazolo [1,5-a]pyridin-6-yl]-phenol;
4-Chloro-3-[2-(2-hydroxymethyl-pyridin-4-ylamino)-[1,2,4]triazolo[1,5-a]pyridin-6-yl]-phenol;
4-Chloro-3-[2-(6-hydroxymethyl-pyridin-3-ylamino)-[1,2,4]triazolo[1,5-a]pyridin-6-yl]-phenol;
3-[2-(3,5-Bis-hydroxymethyl-phenylamino)-quinazolin-6-yl]-4-chloro-phenol;
4-Chloro-3-[2-(pyridin-3-ylamino)-quinazolin-6-yl]-phenol;
4-Chloro-3-[2-(1H-indol-6-ylamino)-[1,2,4]triazolo[1,5-a]pyridin-6-yl]-phenol;
4-[6-(2-Chloro-5-hydroxy-phenyl)-[1,2,4]triazolo[1,5-a]pyridin-2-ylamino]-pyridin-2-ol;
4-Chloro-3-[2-(2-methoxy-pyridin-4-ylamino)-[1,2,4]triazolo[1,5-a]pyridin-6-yl]-phenol;
4-Chloro-3-[2-(5-hydroxymethyl-pyridin-3-ylamino)-quinazolin-6-yl]-phenol;
4-Chloro-3-{2-[1-(2-hydroxy-ethyl)-1H-pyrazol-4-ylamino]-[1,2,4]triazolo[1,5-a]pyridin-6-yl}-phenol;
4-Chloro-3-[2-(1-methyl-1H-pyrazol-4-ylamino)-[1,2,4]triazolo[1,5-a]pyridin-6-yl]-phenol;
4-Chloro-3-{2-[6-(2-pyrrolidin-1-yl-ethoxy)-pyridin-3-ylamino]-[1,2,4]triazolo[1,5-a]pyridin-6-yl}-phenol;
4-Chloro-3-{2-[5-(2-pyrrolidin-1-yl-ethoxy)-pyridin-3-ylamino]-[1,2,4]triazolo[1,5-a]pyridin-6-yl}-phenol;
4-Chloro-3-(2-{6-[4-(2-hydroxy-ethyl)-piperazin-1-yl]-2-methyl-pyrimidin-4-ylamino}-[1,2,4]triazolo[1,5-a]pyridin-6-yl)-phenol;
4-Chloro-3-(2-{3-[4-(2-hydroxy-ethyl)-piperazin-1-yl]-5-methyl-phenylamino}-[1,2,4]triazolo[1,5-a]pyridin-6-yl)-phenol;
4-Chloro-3-[2-(3,4,5-trimethoxy-phenylamino)-[1,2,4]triazolo[1,5-a]pyridin-6-yl]-phenol;
4-Chloro-3-{2-[3-(2-hydroxy-ethyl)-3H-benzoimidazol-5-ylamino]-[1,2,4]triazolo[1,5-a]pyridin-6-yl}-phenol;
4-Chloro-3-[2-(pyridin-3-ylamino)-[1,2,4]triazolo[1,5-a]pyridin-7-yl]-phenol;
4-Chloro-3-{2-[2-(2-pyrrolidin-1-yl-ethoxy)-pyridin-4-ylamino]-[1,2,4]triazolo[1,5-a]pyridin-6-yl}-phenol;
3-[2-(3,5-Bis-hydroxymethyl-phenylamino)-[1,2,4]triazolo[1,5-a]pyridin-7-yl]-4-chloro-phenol;
3-[2-(3,4-Bis-hydroxymethyl-phenylamino)-[1,2,4]triazolo[1,5-a]pyridin-6-yl]-4-chloro-phenol;
4-Chloro-3-[2-(3,4,5-trimethoxy-phenylamino)-quinazolin-6-yl]-phenol;
2-{4-[6-(2-Chloro-5-hydroxy-phenyl)-[1,2,4]triazolo[1,5-a]pyridin-2-ylamino]-pyrazol-1-yl}-1-piperazin-1-yl-ethanone;
4-Chloro-3-(2-{2-[4-(2-hydroxy-ethyl)-piperazin-1-yl]-pyridin-4-ylamino}-[1,2,4]triazolo [1,5-a]pyridin-6-yl)-phenol;
4-Chloro-3-{7-methoxy-2-[4-(2-pyrrolidin-1-yl-ethoxy)-phenylamino]-[1,2,4]triazolo[1,5-a]pyridin-6-yl}-phenol;
4-Chloro-3-[2-(6-methoxy-pyridin-3-ylamino)-quinazolin-6-yl]-phenol;
4-Chloro-3-(2-{4-[2-(1-oxy-pyrrolidin-1-yl)-ethoxy]-phenylamino}-[1,2,4]triazolo[1,5-a]pyridin-6-yl)-phenol;
4-Chloro-3-[2-(1H-indol-6-ylamino)-quinazolin-6-yl]-phenol;
4-Chloro-3-[2-(2-hydroxymethyl-pyridin-4-ylamino)-quinazolin-6-yl]-phenol;
1-(2-{5-[6-(2-Chloro-5-hydroxy-phenyl)-[1,2,4]triazolo[1,5-a]pyridin-2-ylamino]-pyridin-2-yloxy}-ethyl)-pyrrolidin-2-one;
4-Chloro-3-{2-[1-(2-hydroxy-ethyl)-1H-benzoimidazol-5-ylamino]-quinazolin-6-yl}-phenol;
4-Chloro-3-(2-{3-[4-(2-hydroxy-ethyl)-piperazin-1-yl]-5-methyl-phenylamino}-quinazolin-6-yl)-phenol;
4-Chloro-3-(2-{3-[4-(2-hydroxy-ethyl)-piperazin-1-yl]-5-hydroxymethyl-phenylamino}-[1,2,4]triazolo[1,5-a]pyridin-6-yl)-phenol;
Benzoic acid 4-chloro-3-(2-{3-[4-(2-hydroxy-ethyl)-piperazin-1-yl]-5-methyl-phenylamino}-[1,2,4]triazolo [1,5-a]pyridin-6-yl)-phenyl ester;
Benzoic acid 4-chloro-3-(2-{3-[4-(2-hydroxy-ethyl)-piperazin-1-yl]-5-methyl-phenylamino}-quinazolin-6-yl)-phenyl ester;
4-Chloro-3-(2-{3-[4-(2-hydroxy-ethyl)-piperazin-1-yl]-5-hydroxymethyl-phenylamino}-quinazolin-6-yl)-phenol;
and any prodrug thereof.

40. A medicament comprising a compound of formula (I) as defined in claim 23.

41. A pharmaceutical composition comprising a compound of formula (I) according to claim 23, or a prodrug of a compound of formula (I), and also at least one pharmaceutically acceptable excipient.

42. A method for treating, preventing or ameliorating retinal oedema, macular oedema, age-related macular degeneration, ischemia-related retinal vascular leakage, diabetic retinopathy, retinal vein occlusion, or vitreoretinal disease, said method comprising the administration of a therapeutically effective amount of one or more compounds of formula (I) as defined in claim 23 to a subject in need thereof.

43. A method for inhibiting Src kinase, said method comprising the administration of a Src kinase antagonist of formula (I) as defined in claim 23.
